(19) United States
(12) Patent Application Publication
(10) Pub. No.: US 2025/0063654 A1
(43) Pub. Date: Feb. 20, 2025

(54) STRETCHABLE, FABRIC SENSOR, WEARABLE ELECTRONIC DEVICE INCLUDING THE SAME, AND METHOD OF MAKING THE SAME

(71) Applicant: Yale University, New Haven, CT (US)

(72) Inventors: LINA SANCHEZ-BOTERO, New Haven, CT (US); ANJALI AGRAWALA, New Haven, CT (US); REBECCA KRAMER-BOTTIGLLO, Hamden, CT (US)

(21) Appl. No.: 18/718,839

(22) PCT Filed: Sep. 13, 2023
(86) PCT No.: PCT/US23/32593

§ 371 Date: Jun. 12, 2024

Related U.S. Application Data
(60) Provisional application No. 63/406,925, filed on Sep. 15, 2022.

(51) Int. Cl. H05K 1/03; D03D 1/00; G06F 3/01; H05K 3/02

(52) U.S. Cl. CPC H05K 1/038; D03D 1/0088; G06F 3/017; H05K 1/036; H05K 3/022; D10B 2401/16; D10B 2401/18; H05K 2201/029

(57) ABSTRACT

A fabric capacitive strain sensor integrated into everyday clothing to measure human motions. The sensor is made of thin layers of breathable fabrics and exhibits high strains, excellent cyclic stability, and high water vapor transmission rates, which allows for sweat evaporation. The sensor's functionality is evaluated under conditions similar to those experienced on the surface of the human body (35° C. and 90±2% relative humidity) and after washing with fabric detergent. The fabric sensor shows stable capacitance at excitation frequencies up to 1 MHz.

### CROSS-REFERENCE TO RELATED APPLICATIONS

The present application claims the benefit of U.S. Provisional Application No. 63/406,925, the subject matter of which is herein incorporated by reference in its entirety.

### STATEMENT OF FEDERALLY SPONSORED RESEARCH

This invention was made with government support under 1954591 awarded by the National Science Foundation. The government has certain rights in the invention.

### FIELD OF THE INVENTION

The present invention relates generally to a fabric capacitive strain sensor for integration into clothing and wearable devices to measure human motions. The sensor is made of thin layers of breathable fabrics and exhibits high strains, excellent cyclic stability, and high water vapor transmission rates.

### BACKGROUND OF THE INVENTION

In recent years, a growing interest in wearable electronic devices has turned continuous health monitoring into an achievable and mainstream concept, with revolutionary implications for human health, safety, and performance. While wearable commercial devices currently allow users to monitor physiological data such as heart rate, skin conductance, and respiration patterns, there remain a need for noninvasive human motion monitoring systems capable of capturing the body strains involved in everyday activities. In addition, there also remains a need for noninvasive human motion monitoring systems that are more comfortable for the user and can be used in a home or clinic environment for real-time diagnosing and/or monitoring of conditions such as sleep-based movement disorders (i.e., limb movement disorders, restless leg syndrome, REM-sleep behavior disorder, etc.) and other chronic conditions, and that can also be used for post-op recovery and remote health monitoring.

In situ collection of human motion data is crucial for advancing the current state of a broad range of disciplines including human-robot interactions, virtual reality, sports performance, and personalized health monitoring and rehabilitation. Most commonly, human motion data is collected via use of optical, electromagnetic, and inertial measurement unit (IMU) motion capture systems. Although optical motion capture systems, consisting of multiple cameras oriented around a subject, are broadly considered the gold standard for high accuracy, these systems are susceptible to measurement errors and loss of analyzable data due to occluded lines of sight. Such setups limit the spatial volume of the analysis and necessitate controlled laboratory environments.

In contrast, electromagnetic motion capture systems employ sensors that provide measurements without requiring lines of sight. However, this method is limited to use in controlled settings because electromagnetic interference from the surrounding environment can lead to measurement errors.

Like electromagnetic motion capture sensors, IMUs can be mounted onto the subject and provide measurements using only the onboard gyroscope and accelerometer. While the use of IMUs is not limited to controlled laboratory settings, they exhibit positional drift in long-term measurements and are typically made of rigid components, which limit user comfort.

The development of soft strain sensors has shown promise for circumventing the existing challenges of traditional motion capture systems and enabling unobtrusive integration of human motion monitoring. Typically, such soft strain sensors employ conductive composites with fillers such as carbon black, carbon nanotubes, metallic nanoparticles, silver nanowires, graphene, liquid metals, and/or ionic fluids to create electrodes. Silicone-based elastomers such as polydimethylsiloxane (PDMS), biodegradable compostable elastomers (a commercial product of which is available from Eco-Flex under the tradename Ecoflex®), and high performance silicone rubbers (a commercial product of which is available from Smooth-On, Inc. under the tradename Dragon Skin™) usually serve as both insulating host materials and highly stretchable substrates. The use of such compliant materials allows soft sensors to conform to curvilinear surfaces (e.g., elbows, knees) and withstand the everyday skin deformations of human joints (in the range of 40%-55% strain) without impeding natural motion.

Most soft strain sensors transduce uniaxial mechanical deformation into a change in either electrical resistance or capacitance. Resistive strain sensors have demonstrated higher sensitivities than capacitive strain sensors overall, but many also show limited electromechanical robustness, hysteresis, and low sensing stability due to crack formation and mechanical damage at high strains. Since the sensor response of most capacitive sensors relies on the overlapping area of electrodes, capacitive sensors generally show more linear and stable behavior, both of which are particularly important in human motion monitoring.

Although there are many capacitive strain sensors known in the art, there remain several key ongoing challenges in the field regarding breathability, maximum measurement frequency, and sensor-garment integration. Based thereon, it would be desirable to provide a wearable capacitive sensor that addresses these identified limitations.

Wearable strain sensors for movement tracking are a promising paradigm to improve clinical care for patients with neurological or musculoskeletal conditions, with further applicability to athletic wear, virtual reality, and next-generation game controllers. Clothing-like wearable strain sensors can support these use cases, as the fabrics used for clothing are generally lightweight and breathable, and interface with the skin in a manner that is mechanically and thermally familiar.

Despite the prevalence of elastomer-based sensors, such sensors compromise thermophysiological and skin sensorial comfort due to the low air permeability and water vapor transmission of elastomers. Recently, the use of fabrics to improve sensor comfort has been explored. For example, a soft parallel-plate capacitor constructed using conductive fabric as electrodes and a silicone layer as the dielectric material has been introduced (A. Atalay et al., “Composite Capacitive Strain Sensors for Human Motion Tracking,” Advanced Materials Technologies, Vol. 2, 1700126 (2017). Although this sensor uses fabric as the outer exposed (conductive) layers, the internal silicone dielectric layer limits the overall breathability of the sensor. Another capacitive sensor (Park et al., “Sim-To-Real Transfer Learning Approach for Tracking Multi-DOF Ankle Motions Using Soft Strain Sensors,” IEEE Robotics and Automation Letters, Vol. 5, No. 2, pp. 3525-3532 April 2020) uses silicone to bind fabric layers such that fabrics serve as both the electrode and dielectric materials. In both of these works, as well as most wearable electronics and sensor literature, the breathability of the sensor materials was not characterized.

Resistive sensors operate based on a simple increase in electrical resistance with increasing strain (and corresponding decreasing cross-sectional area). Because the output is simple resistance (or voltage), the signal conditioning is relatively easy. The major issue with resistive sensors, especially for high-strain applications, is the coupling between the electrical and mechanical material behaviors. All elastic materials (needed for high strains) show non-linear stress versus strain dependencies and mechanical hysteresis, and these characteristics result in non-linear resistance vs strain dependencies and electrical hysteresis for resistive sensors. Furthermore, resistive fabric sensors tend to be especially sensitive to the shifting contacts between individual fibers in the fabric, which means a constantly changing the electrical pathway through a conductive fabric sensor.

Capacitive sensors operate via a changing capacitance between two electrodes separated by a dielectric material, which decreases in thickness with increasing strain, thus bringing the electrodes closer together with increasing strain or further apart with decreasing strain. The output is capacitance, which needs to be transduced into a voltage, and therefore the signal conditioning circuits are more complex. However, capacitive sensors decouple the mechanical and electrical behaviors. So, while elastic capacitive sensors still show nonlinear mechanical behavior and hysteresis, the electrical behavior is typically linear and shows no hysteresis. This linearity and lack of hysteresis is one of the main advantages of capacitive sensors.

Capacitive sensors are made of multiple layers that must be well-bonded. However, fabric layers do not inherently bond to one another and stacked layers of unaltered fabric would slide over one another easily. Researchers have responded to this challenge by using silicones or other adhesives as the dielectric layer, which enables good bonding between two conductive fabric electrodes. But, by using silicone layers in the sensor, it is no longer fully fabric. That is, while the surface may still feel like a fabric, making it soft and familiar to the touch, but the sensor component will not have air permeability or a water vapor transmission rate (WVTR) that meets the standard of wearability.

### SUMMARY OF THE INVENTION

In order to overcome these deficiencies, the inventors of the present invention have developed a fully fabric capacitive sensor that uses a porous, breathable fabric adhesive to bond the fabric electrode and dielectric layers. In addition, the fabric capacitive sensor described herein is designed to meet the air permeability and WVTR requirements of wearables.

In addition, it has been established that electrode resistance can affect the maximum frequency at which capacitance can be accurately measured, with characteristic frequencies around 5 kHz. However, as described herein, the inventors of the present invention have discovered that it is possible to achieve stable capacitance measurements up to 1 MHz using single-board microcontrollers (i.e., standard plug and play Arduino or similar hardware) using the fabric capacitive sensors described herein. The fidelity of the sensor response at high frequencies indicates its suitability for broader translation into soft robotics applications.

In addition, continuous enhancement in wearable technologies has led to several innovations in the healthcare, virtual reality, and robotics sectors. One form of wearable technology is wearable sensors for kinematic measurements of human motion. However, measuring the kinematics of human movement is a challenging problem as wearable sensors need to conform to complex curvatures and deform without limiting the user's natural range of motion. In fine motor activities, such challenges are further exacerbated by the dense packing of several joints, coupled joint motions, and relatively small deformations.

The design, fabrication, and characterization of a thin, breathable sensing glove capable of reconstructing fine motor kinematics is also described. The fabric glove features capacitive sensors made from layers of conductive and dielectric fabrics, culminating in a non-bulky and discrete glove design.

Wearable systems invite tangible interactions with robots in contexts such as virtual reality (VR), augmented reality (AR), and teleoperation. Recent developments in wearable technology have incorporated soft sensing mechanisms for kinematic measurements. However, human motion invokes challenges associated with complex curvatures and form factors to which rigid systems do not comply. Soft sensors present a promising solution for the challenges associated with measuring human kinematics due to their deformability and robustness under strain. Notwithstanding the advantages of soft sensors, fine motor joints present unique challenges for kinematic estimation due to their small angular displacements, coupled joint motions, and number of joints in close proximity.

Many sensing gloves have been designed for applications ranging from VR to robotics. However, current solutions include bulky mechanical components and complex wiring systems that impede motion and cause discomfort. Other gloves include elastomer-based sensors that prevent the overall breathability and washability of the glove. A wearable sensing glove made entirely of fabrics to minimize the amount of material required for sensing, while maintaining properties traditional to garments is desired.

Fabric-based electronics allow the tight coupling of technology into traditional garments. As described in detail herein, fabric-based strain sensors can be easily integrated into garments while maintaining properties native to fabrics, including breathability and washability. While fabric-based technologies have been implemented toward fine motor motion monitoring, limitations in fabric-based sensing gloves still remain with respect to the quantification of accuracy, minimalistic design, and the conservation of properties inherent to textiles.

The fabrication process of a fabric sensing glove and the glove's ability to accurately estimate joint angles compared to ground truth from a motion capture system is described herein. The use of the fabric sensor in the form of a glove improves upon current solutions that include bulky, cumbersome, and uncomfortable components.

It is an object of the present invention to provide a fabric capacitive strain sensor.

It is another object of the present invention to provide a fabric capacitive strain sensor for integrating into clothing and wearable devices.

It is still another object of the present invention to provide a fabric capacitive strain sensor that is breathable.

It is still another object of the present invention to provide a fabric capacitive strain sensor that meets the air permeability and water vapor transmission rate requirements of wearable devices.

It is yet another object of the present invention to provide a fabric capacitive strain sensor to measure human motion.

To that end, in one embodiment, the present invention relates generally to a stretchable electronic sensor, wherein the stretchable electronic sensor comprises:
two or more stretchable fabric electrodes separated by at least one stretchable dielectric layer, wherein the two or more stretchable fabric electrodes are secured to the at least one dielectric layer with two or more adhesive layers.

In another embodiment, the present invention also relates generally to a stretchable electronic sensor, wherein the stretchable electronic sensor comprises, in order:
a first outer stretchable conductive fabric layer;
a first inner stretchable dielectric layer;
an inner stretchable conductive fabric layer;
a second inner stretchable dielectric layer; and
a second outer stretchable conductive fabric layer;
wherein an adhesive layer is sandwiched between each of the layers of the stretchable electronic sensor, wherein the adhesive layer comprises an adhesive film, and wherein the layers of the stretchable electronic sensor are joined together to form the stretchable electronic sensor.

The present invention also relates generally to a wearable electronic device comprising the stretchable electronic sensor described herein,
wherein the wearable electronic device comprises a stretchable garment,
wherein the stretchable garment comprises an outer surface and an inner surface;
wherein the stretchable electronic sensor is coupled to or integrated into the stretchable garment at a location where it is desirable to monitor motion of a user, wherein the stretchable garment comprises one of the first inner stretchable dielectric layer or the second inner stretchable dielectric layer of the stretchable electronic sensor; and
wherein one of the first outer stretchable conductive fabric layer and the second outer stretchable conductive layers is contactable with the user's skin.

### BRIEF DESCRIPTION OF THE FIGURES

Features and aspects of embodiments are described below with reference to the accompanying drawings, in which elements are not necessarily depicted to scale, and in certain views, parts may have been exaggerated or removed for purposes of clarity.

Exemplary embodiments of the present disclosure are further described with reference to the appended figures. It is to be noted that the various features, steps and combinations of features/steps described below and illustrated in the figures can be arranged and organized differently to result in embodiments which are still within the scope of the present disclosure.

To assist those of ordinary skill in the art in making and using the disclosed assemblies, systems and methods, reference is made to the appended figures, wherein:

FIG. 1 depicts the overview of the fabric sensor in which (a) the fabric sensor has a three-layer construction, (b) the fabric sensor has a five-layer constructure, and (c) where the fabric sensors are integrated into garments for human motion monitoring.

FIG. 2(a) depicts dimensions of one example of a dog-bone shaped sensor in accordance with the present invention. FIGS. 2(b) and 2(c) depicts schematic representations of 3- and 5-layers of the sensors.

FIG. 3(a) depicts another view of the dog-bone shaped sensor of the present invention and FIG. 3(b) depicts changes in the sensor dimension during tensile stretch, resulting in a measurable change in capacitance.

FIGS. 4(a)-(c) depict a schematic image of a warp-knit tricot structure conductive fabric along with optical images of the front and back of the conductive fabric. Both the conductive fabric and the dielectric nylon fabric exhibit a warp-knit tricot structure, which is textured on one side and smooth on the other. FIGS. 4(d)-(f) depict a schematic image of a weft-knit jersey structure conductive fabric along with optical images of the front and back of the conductive fabric. Both polyester and cotton fabrics exhibit a weft-knit jersey structure, where each loop in the vertical direction is “hung” on top of the previous loops in the horizontal direction.

FIG. 5 depicts SEM images of a pristine adhesive film in accordance with the present invention.

FIG. 6 depicts sensor material breathability and morphology characteristics of various conductive fabrics in accordance with the present invention. FIGS. 6(a) and 6(b) depicts air permeability and water vapor transmission rate of bare and laminated fabrics (i.e., fabrics coated with the adhesive). An average of three samples is shown for each. FIGS. 6(c)-(f) depicts SEM images of conductive nylon, nylon, polyester, and cotton fabrics and, from left to right, the front side of the bare fabrics, fiber morphologies, and laminated fabrics.

FIG. 7 depicts electromechanical characterizations of five-layer fabric sensors. FIGS. 7(a)-(c) depict the average relative change in capacitance as a function of strain for five sensors with dielectric nylon, polyester, and cotton, and three sensitivity regimes are shown. FIGS. 7(d)-(f) depict 5000 strain cycles to ≈60% for a representative sensor with dielectric for nylon, polyester, and cotton. FIGS. 7(g)-(i) depict the average change in capacitance as a function of frequency for five sensors with dielectric nylon, polyester, and cotton.

FIG. 8 depicts electromechanical characterizations of three-layer fabric sensors. FIGS. 8(a)-(c) depict the cyclic stability of a representative 3-layer sensor with dielectric nylon, polyester, (c) cotton. FIGS. 8(d)-(f) depict the average change in capacitance as a function of frequency for five 3-layer sensors with dielectric nylon, polyester, and cotton.

FIG. 9 depicts the effect of temperature, humidity, and washing on the electro-mechanical response of five-layer sensors. FIGS. 9(a) and (b) depict the average relative change in capacitance as a function of strain, under varying temperatures and humidities, for five sensors with dielectric nylon and polyester. FIGS. 9(c) and 9(d) depict the effect of washing on the electromechanical response of five sensors with dielectric nylon and polyester.

FIG. 10 depicts a sensory smart garment capable of monitoring the movement of body joints. FIG. 10(a) shows the sensor placement on the knees, elbows, and hips of the garments. FIGS. 10(b) to 10(e) depict photographs and capacitance responses of the sensors during the following human motions: FIG. 10(b) squats (10 cycles), FIG. 10(c) sit-to-stand cycles (10 cycles), FIG. 10(d) step-ups (10 cycles), FIG. 10(e) retrieving an object from the floor (2 cycles).

FIG. 11 is a table that includes data for the average thicknesses and normalized weights of each fabric, bare and laminated with adhesive film.

FIGS. 12(a)-(c) depict relative change in capacitance and sensitivity as a function of strain for 3-layer sensors with dielectric nylon, polyester, and cotton. The average response of five sensors is shown in FIGS. 12(a) and (b) and of three sensors in FIG. 12(c). The discrete points are showing the experimental measurements for the average capacitance plotted against strain, while the black solid lines are showing sensitivity results. The sensitivity values for 3-layer sensors within the strain regions are comparable to those obtained for the 5-layer sensors.

FIGS. 13(a)-(c) depict the relative change in capacitance as a function of strain for both 5-layer and 3-layer sensors with dielectric nylon, polyester, and cotton. The relative change in capacitance for 3-layer sensors within the strain regions are comparable to those obtained for the 5-layer sensors.

FIGS. 14(a)-(c) depict stress as a function of strain for 3- and 5-layer sensors with dielectric nylon, polyester, and cotton. The average response of five sensors is shown for each.

FIG. 15 depicts the average plastic deformation of five 3- and 5-layer nylon, polyester, and cotton sensors after the first 10 cycles of 100% strain.

FIG. 16 depicts the response time of a representative 5-layer nylon sensor in response to a step-like strain with a rate of 5 mm/s. The average response time of five 5-layer nylon sensors was found to be 179 ms with a standard deviation of 46 ms.

FIG. 17 depicts the average relative change in capacitance as a function of strain during 10 cycles of loading and unloading for five 5-layer nylon sensors. Data were taken in ambient lab conditions: temperature=23±1° C. and relative humidity=29±2%.

FIG. 18 depicts the average relative change in capacitance as a function of strain for five 5-layer nylon sensors at strain rates of 1 mm/s, 5 mm/s, and 10 mm/s. Data was taken in the ambient lab conditions: temperature=23±1° C. and relative humidity=29±2%. Data was collected from the same sensors in sequence of 5, 1, then 10 mm/s.

FIGS. 19(a) and (b) depict the effect of temperature and humidity on the electromechanical response of 5-layer sensors with dielectric nylon and polyester. The discrete points are showing the experimental measurements for the average capacitance of five sensors plotted against strain, while the solid lines are showing the gauge factor/sensitivity results.

FIGS. 20(a) and (b) depict the effect of temperature and humidity on the electromechanical response of five 5-layer sensors with dielectric nylon and polyester.

FIGS. 21(a)-(d) depict the effect of temperature and humidity on the electromechanical response of five 3-layer sensors with dielectric (nylon and polyester.

FIGS. 22(a) and (b) depict the effect of humidity and temperature on the average capacitance of five 5-layer sensors with dielectric nylon or polyester at 0% strain.

FIGS. 23(a) and (b) depict the effect of temperature and humidity on the electromechanical response of 5-layer sensors with dielectric nylon and polyester. Dashed lines are the fittings of the predicted capacitance based on Equation 19.

FIG. 24(a) depicts a photograph of a sensorized glove in accordance with one aspect of the present invention with the capacitive sensors along the middle phalanges of each finger to capture the movement of the metacarpophalangeal and proximal interphalangeal joints. FIGS. 24(b)-(g) detail fabrication steps of a sensorized glove in accordance with the present invention.

FIG. 25 depicts average normalized change in capacitance vs. strain for five fabric sensors. The capacitance of the sensors was recorded using the materials testing system (Instron® 3345) and an LCR meter (E4980AL, Keysight Technologies) at an excitation frequency of 1 kHz.

FIG. 26 depicts average normalized change in capacitance vs. bending angles of the PIP (top) and MCP joints (bottom) of the thumb and the pointer finger. Error cloud represents one standard deviation.

FIG. 27 depicts calibration of average relative change in capacitance vs. bending angles of the PIP (top row) and MCP joints (bottom row) of the thumb shown in red, index finger shown in blue, middle finger shown in yellow, ring finger shown in green, and pinky finger shown in pink. The insets depict the reference axis (zero-degree axis) defined for each joint bending angle to serve as ground truth.

FIGS. 28(a)-(d) depict plots of ground truth and measured pose angles for (a-b) middle MCP joint, and (c-d) middle PIP joint.

FIG. 29 depicts post reconstruction (top row) and corresponding photographs (bottom row) depicting the intended hand positions for the letters spelling the word “Yale” in American Sign Language.

Like parts are marked throughout the specification and drawings with the same reference numerals, respectively.

### DETAILED DESCRIPTION OF THE PREFERRED EMBODIMENTS

To maximize wearer comfort and safety, and encourage real-world usage, the inventors of the present invention sought to create a reliable strain sensor made of entirely conductive and non-conductive fabrics bound together with thin films of breathable thermoplastic fabric adhesive. The fabric sensor of the instant invention can be coupled to, integrated into, or otherwise directly embedded into commercial activewear, clothing, garments and/or and other wearable devices. In addition, the fabric sensors can also use the garment or wearable device itself as the dielectric layer of the sensors, thus overcoming existing challenges of bulky attachment modes and sensor detachment and/or slippage.

By using fabrics and porous layers that offer a unique combination of flexibility, stretchability, and breathability, sensor wearability and user tactile comfort (as measured by air permeability and water vapor transmission) are prioritized in a way that existing elastomer-based sensors do not.

Sensor performance is characterized using three common fabrics (i.e., cotton, polyester, and nylon) as the dielectric materials to demonstrate the respective advantages of each. In addition, the sensor's cyclic stability, frequency dependence, electromechanical response to temperature and humidity, and washability are evaluated. Along with its functional benefits, the fabric sensors are fabricated using a simple, highly reproducible, and low-cost stacked assembly method, which allows for their seamless integration into commercial clothing and other wearable devices to facilitate the collection of reliable human motion data.

As described herein, the present invention is directed to a fabric capacitive strain sensor that can be integrated into everyday clothing to measure human motions. The sensor is made of thin layers of breathable fabrics and exhibits high strains (>90%), excellent cyclic stability (>5000 cycles), and high water vapor transmission rates (>30 g/h m²), the latter of which allows for sweat evaporation, an essential parameter of comfort. The sensor's functionality was analyzed under conditions similar to those experienced on the surface of the human body (35° C. and 90±2% relative humidity) and after washing with fabric detergent. As described in detail herein, the fabric sensors of the invention show stable capacitance at excitation frequencies up to 1 MHz, facilitating their low-cost implementation. With the prioritization of breathability (air permeability and water vapor transmission), the fabric sensor design presented herein paves the way for future comfortable, unobtrusive, and discrete sensory clothing and wearable devices that exhibit increased comfort.

As used herein, “a,” “an,” and “the” refer to both singular and plural referents unless the context clearly dictates otherwise.

As used herein, the term “about” refers to a measurable value such as a parameter, an amount, a temporal duration, and the like and is meant to include variations of +/−15% or less, preferably variations of +/−10% or less, more preferably variations of +/−5% or less, even more preferably variations of +/−1% or less, and still more preferably variations of +/−0.1% or less of and from the particularly recited value, in so far as such variations are appropriate to perform in the invention described herein. Furthermore, it is also to be understood that the value to which the modifier “about” refers is itself specifically disclosed herein.

As used herein, spatially relative terms, such as “beneath,” “below,” “lower,” “above,” “upper” and the like, are used for ease of description to describe one element or feature's relationship to another element(s) or feature(s) as illustrated in the figures. Spatially relative terms may be intended to encompass different orientations of the device in use or operation in addition to the orientation depicted in the figures.

It is further understood that the terms “front” and “back” are not intended to be limiting and are intended to be interchangeable where appropriate.

As used herein, the terms “comprises” and/or “comprising,” specify the presence of stated features, integers, steps, operations, elements, and/or components, but do not preclude the presence or addition of one or more other features, integers, steps, operations, elements, components, and/or groups thereof.

In one embodiment, the present invention relates generally to a stretchable electronic sensor, wherein the stretchable electronic sensor comprises:
two or more stretchable fabric electrodes separated by at least one stretchable dielectric layer, wherein the two or more stretchable fabric electrodes are secured to the at least one dielectric layer with two or more adhesive layers, wherein the stretchable electronic sensor has an air permeability of greater than 50 l/m²s, preferably between 50 and 1,000 l/m²s, or a water vapor permeability greater than about 30 g/m²h, preferably between about 30 and about 150 g/m²h.

In another embodiment, the present invention also relates generally to a stretchable electronic sensor, wherein the stretchable electronic sensor comprises, in order:
a first outer stretchable conductive fabric layer;
a first inner stretchable dielectric layer;
an inner stretchable conductive fabric layer;
a second inner stretchable dielectric layer; and
a second outer stretchable conductive fabric layer;
wherein an adhesive layer is sandwiched between each of the layers of the stretchable electronic sensor, wherein the adhesive layer comprises an adhesive film, wherein the adhesive film preserves porosity between adjacent layers, and wherein the layers of the stretchable electronic sensor are joined together to form the stretchable electronic sensor.

In one embodiment, the layers are joined together by laminating the layers using at least one of heat or pressure.

The stretchable electronic sensor also comprises a ground wire or layer connected to the first outer stretchable conductive fabric layer or the second outer stretchable conductive layer and a second wire or layer connected to the inner stretchable conductive fabric layer.

The stretchable electronic sensor also preferably comprises a non-elastic tab at a first end and a second end of the stretchable electronic sensor, such that the surface area between the non-elastic tabs define the area of stretchability of the stretchable electronic sensor.

In one embodiment, the inner stretchable conductive fabric layer has a surface area that is less than the first inner stretchable dielectric layer or the second inner stretchable dielectric layer. The first outer stretchable conductive fabric layer, the second outer stretchable conductive fabric layer and the inner stretchable conductive fabric layer are knit fabric or woven fabrics. In one embodiment, the knit fabrics and woven fabrics are selected from the group consisting of conductive polyester, conductive nylon, conductive natural fibers, including conductive cotton and cotton blends, conductive polypropylene, knit or woven fabrics coated with a conductive ink or other conductive layer or material, and combinations of any of the foregoing.

In one embodiment, the first outer stretchable conductive fabric layer, the second outer stretchable conductive fabric layer and the inner stretchable conductive fabric layer have a surface resistivity of less than 10 Ω/sq, more preferably less than 1 Ω/sq.

In one embodiment, the stretchable conductive fabric layers comprise a fabric material woven or knitted from fibers coated with conductive nanoparticles and/or nanofibers. In one embodiment the fibers comprise natural fibers or polymer fibers, wherein the polymer fibers comprise nylon, polyester, polyurethane (including Lycra® and spandex), and combinations of one or more of the foregoing, In one embodiment, the conductive nanoparticles and/or nanofibers are selected from the group consisting of silver, gold, copper, zinc oxide, aluminum, tin, nickel, carbon black, carbon nanofibers, carbon nanotubes, graphite, graphene, iron and iron compounds (including iron compounds and alloys such as carbonyl iron, FeHO₂, NdFeB, etc.), and combinations thereof.

The adhesive layer may be any adhesive layer that can be used to adhere the various fabric layers and provide the desired properties of porosity, breathability, air permeability and water vapor transmission rate. In one embodiment, the adhesive layer comprises a thermoplastic adhesive which may be broadly defined to be any polymer which softens and melts when heated. In one embodiment, the thermoplastic adhesive is a thermoplastic film or web. Suitable thermoplastic films and webs include hot melt adhesive films, including, but not limited to, ethylene-vinyl acetate, polyolefin-based hot melt adhesives, polyamides, thermoplastic polyurethane, epoxies, polyvinyl acetate, polyimides, polyacrylates and polyesters. One example of a suitable adhesive layer is a thermoplastic polyurethane fabric tape.

Typical air permeability values for clothing may range from 1 l/m²s in the case of garments with impermeable membranes up to 1,000 l/m²s for highly permeable garments such as unlined fleece garments. In one embodiment, the stretchable electronic sensor has an air permeability that is greater than 50 l/m²s or greater than 75 l/m²s or greater than 100 l/m²s. In another embodiment, the stretchable electronic sensor has an air permeability in the range of about 50 to about 1,000 l/m²s, more preferably about 100 to about 500 l/m²s.

In one embodiment, the stretchable electronic sensor has a water vapor permeability that is greater than about 30 g/m²h, or greater than about 35 g/m²h, preferably between about 30 and about 150 g/m²h, more preferably between about 35 to about 120 g/m²h or between about 35-45 g/m²h or between about 38-41 g/m²h.

In one embodiment, the stretchable electronic sensor described herein is coupled to or integrated into a wearable electronic device. The wearable electronic device comprises a stretchable garment comprising an outer surface and an inner surface and at least one stretchable electronic sensor is coupled to, integrated into, embedded or otherwise embedded into the wearable electronic device or garment at a location where it is desirable to monitor motion of a user. The stretchable electronic sensor is coupled to or integrated into the stretchable garment so that the stretchable garment comprises one of the first inner stretchable dielectric layer or the second inner stretchable dielectric layer of the stretchable electronic sensor and one of the first outer stretchable conductive fabric layer and the second outer stretchable conductive layers is contactable with the user's skin.

The wearable electronic device is configured to apply compression to the stretchable electronic sensor so as to maintain contact between the stretchable electronic sensor and the user's skin. In one embodiment, the compression is provided by the stretchable garment.

The stretchable garment or wearable electronic device may be selected from the group consisting of modular knee sleeves, modular ankle sleeves, modular elbow sleeves, gloves, leggings, tights, shirts, unitards and combinations of one or more of the foregoing. Other stretchable garments and wearable electronic devices may also be usable in the present invention so long as they can be configured to provide compression to one or more stretchable electronic sensors incorporated therein.

The wearable electronic device may comprise one or more stretchable electronic sensors, wherein each stretchable electronic sensor is integrated into the stretchable garment at a location where it is desired to monitor motion of a user. The stretchable electronic sensor also comprises a ground wire or layer connected to the first or second outer stretchable conductive fabric layer and a second wire or layer connected to the inner stretchable conductive fabric layer. The ground wire or layer and second wire or layer are coupled to a controller to receive signals from the stretchable electronic sensor and measure and monitor capacitive response resulting from the motion of the user. In one embodiment, the controller may comprise a stretchable circuit board such as described in U.S. Pat. Pub. No. 2021/0410283 to Bottiglio et al., the subject matter of which is herein incorporated by reference in its entirety.

In one embodiment, the present invention also relates generally to a method of making a stretchable electronic sensor that is capable of being integrated into a wearable electronic device, the method comprising the steps of:
a) sandwiching an adhesive film between a first stretchable conductive fabric layer and a first stretchable dielectric layer and joining the first stretchable conductive fabric layer to the first stretchable dielectric layer; and
b) sandwiching an adhesive film between the first stretchable dielectric layer and a second stretchable conductive fabric layer and joining the first stretchable dielectric layer to the second stretchable conductive fabric layer,
wherein the adhesive film preserves porosity between adjacent layers.

In one embodiment, the method further comprises the steps of:
c) sandwiching an adhesive film between the second stretchable conductive fabric layer and a second stretchable dielectric layer and joining the first stretchable dielectric layer to the second stretchable conductive fabric layer; and
d) sandwiching an adhesive film between the second stretchable dielectric layer and a third stretchable conductive fabric layer and joining the second stretchable dielectric layer to the third stretchable conductive fabric layer,
wherein the adhesive film preserves porosity between adjacent layers.

In one embodiment, the layers are joined together by laminating the layers using at least one of heat or pressure. The temperature at which the layers will be joined together will depend in part on the melting point of the adhesive being used. In one embodiment, the melting point is at least slightly above the melting point of the particular adhesive.

In one embodiment, the second stretchable conductive fabric layer acts as an internal electrode layer, wherein the inner electrode layer is smaller in surface area than the first stretchable conductive fabric layer and/or the third stretchable conductive fabric layer.

A ground wire or layer is connected to the first or third stretchable conductive fabric layer and a second wire or layer is connected to the second stretchable conductive fabric layer.

The first, second and third stretchable conductive fabric layer comprise a conductive knit fabric or a conductive woven fabric that is breathable and washable.

In one embodiment, the present invention also relates generally to a method of making a wearable electronic device comprising a stretchable electronic sensor, wherein the wearable electronic device comprises a stretchable garment, the method comprising the steps of:
a) sandwiching an adhesive film between a first stretchable conductive fabric layer and a first stretchable dielectric layer and joining the first stretchable conductive fabric layer to the first stretchable dielectric layer; wherein the first stretchable dielectric layer extends across approximately 50% of the length of the first dielectric layer;
b) sandwiching an film between the first stretchable dielectric layer and a second stretchable conductive fabric layer and joining the first stretchable dielectric layer to the second stretchable fabric electrode, wherein the second stretchable conductive fabric layer electrode is smaller in surface area than the first stretchable dielectric layer;
c) sandwiching an adhesive film between an inner layer of garment and the first stretchable conductive fabric layer and joining the first stretchable conductive fabric layer to the inner layer of the garment; and
d) sandwiching an adhesive film between the outer layer of the garment and the second conductive fabric layer and joining the second conductive fabric layer to the outer surface of the garment;
wherein the stretchable electronic sensor is integrated into the stretchable garment.

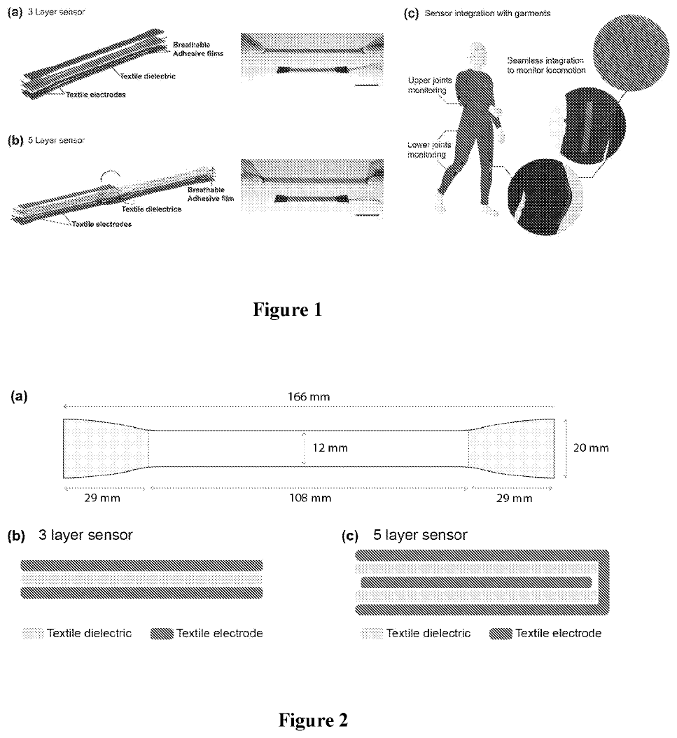

An example of a capacitive strain sensor in accordance with the invention is illustrated in FIGS. 1(a) and 1(b). The capacitive strain sensor comprises conductive fabric electrodes separated by dielectric fabric layers. Each layer is stacked and affixed with breathable adhesive film. Flexible wires are used to interface the sensors with external data acquisition electronics. For example, the external data acquisition electronics may include, a “plug and play system,” for example, an open source software and hardware system such as an Arduino Pro mini and MPR121 Adafruit breakout circuit. FIG. 1(a) illustrates a three-layer sensor configuration while FIG. 1(b) illustrates a five-layer sensor configuration.

In the five-layer configuration, the external electrode is connected to ground, which reduces parasitic capacitance and shields the sensor, therefore making the device more suitable for contact with human skin. The characteristics of the constituent sensor materials and the straightforward fabrication process allow seamless sensor integration into existing knitted garments by the method described herein. The result of this integration is a sensory garment capable of monitoring the movement of body joints as shown in FIG. 1(c).

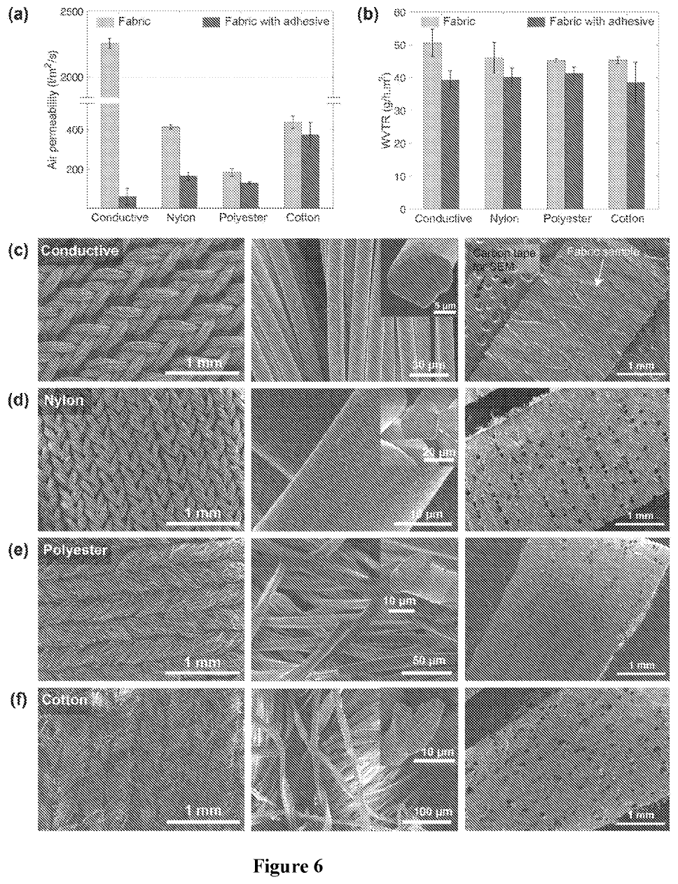

Comfort is one of the most critical components of modern wearable devices. However, this feature is often overlooked in the development of new wearable sensors. For fabrics, tactile and thermophysiological comfort is related to the breathability of the material. Thus, to evaluate the breathability of the sensor, the air permeability and water vapor transmission rate (WVTR) of the sensor's constituent fabrics were tested, both with and without thermoplastic adhesive as shown in FIGS. 6(a) and 6(b).

To demonstrate aspects of the invention, sensors were constructed using knit fabrics, including a medical-grade conductive nylon for the electrodes, and various fabrics for the dielectric layers including, for example, nylon, polyester, and cotton. Air permeability was measured according to the ASTM 737-18 procedure, which determines the volume rate of air flow per unit area of fabric. Both the conductive fabric (shown in FIG. 6(c)) and the dielectric nylon fabric (shown in FIG. 6(d)) exhibited a warp-knit tricot structure as shown in FIG. 4, with air permeability values of 2253.3 and 414 l/m²s, respectively.

Although both the conductive and the dielectric nylon fabrics have the same knit structure, the dielectric nylon fabric exhibits a tighter knit (and thus a lower air permeability) relative to the more open structure of the conductive fabric. In contrast, the polyester (shown in FIG. 6(e)) and cotton (shown in FIG. 6(f)) dielectric fabrics exhibit a weft-knit jersey structure as shown in FIG. 4 with air permeability values of 183 and 439 l/m2s, respectively. Because polyester is the heaviest and thickest of the fabrics tested, it exhibited the lowest air permeability, as shown in FIG. 6(a). Additional fabric characteristics such as fiber hydrophilicity, yarn count, weave or knit structure, fabric thickness, and fabric porosity have also been shown to affect air permeability and water vapor transmission. FIG. 11 provides data for the average thicknesses and normalized weight for each fabric (i.e., conductive nylon and various fabrics for the dielectric layer bare and laminated with adhesive film. Five samples of 1 inch×1 inch were used for each category.

Fabrics coated with the thin film adhesive (a thermoplastic polyurethane fabric tape; morphology as shown in FIG. 5) are referred to herein as “laminated fabrics.” Once adhered to the fabrics, the adhesive visually presents as a porous membrane as shown in the third column of FIG. 6(c)-(f). As a result, the air permeability of the laminated fabrics is reduced compared to their bare fabric counterparts as shown in FIG. 6(a). On average, the air permeability of laminated nylon and polyester is 163 and 129 l/m′s, respectively. These values constitute a respective reduction by ≈60% and ≈30% relative to the bare samples. Laminated cotton exhibited a reduction in permeability of only ≈15%. In contrast, laminated conductive nylon exhibited a reduced permeability of ≈97.2%, which is attributed to the reduced porosity shown in FIG. 6(c).

Although the air permeability of the laminated fabrics was reduced relative to the bare fabrics, the laminated dielectric fabrics all showed air permeabilities greater than 100 l/m²s, which falls within the range of normal clothing breathability. On average, the laminated conductive fabric showed an air permeability slightly less than this value (62 l/m²s). However, the order of attachment of the adhesive to the fabric likely plays a role in the porosity of the fabric-bonded adhesive and air permeability of the overall composite. Thus, it has been found that attaching the adhesive to the dielectric fabric first enables the air permeability of composite layers to be greater than 100 l/m²s.

Water-vapor permeability is another key physical property of fabrics affecting breathability since the loss of water vapor is crucial for the wearer's thermal equilibrium and physiological comfort. Measurements show high WVTRs for all the bare fabrics with average values between 45 and 51 g/h m². All of the laminated fabrics exhibited similarly high WVTR, with average values between 38 and 41 g/h m² as shown FIG. 6(b). The laminated fabrics behave as porous membranes with WVTRs higher than the rate of transepidermal water loss (TEWL) of adult skin under normal conditions (5-10 g/h m²) and within the range of TEWL during sweating (6-66 g/h m²). The samples described herein also have WVTRs higher than those of non-porous 8 μm films and highly porous (45%) 40 μm films of polydimethylsiloxane (5-6 and 20.3 g/h m², respectively), which are elastomers commonly used in wearable devices. Therefore, it can be seen that fabric laminated with the adhesive film has a minimal blocking effect on moisture permeability.

Although the three-layer configuration for capacitive sensors is the most widely used in electrical and robotic applications, it is believed that the five-layer configuration is most suitable for wearable applications in contact with the human skin. In the five-layer configuration, the external electrode acts as an active shield when connected to ground, mitigating parasitic and environmental interference factors and resulting in a high fidelity signal. On the other hand, in the three-layer configuration, while operational in wearable applications, direct skin contact with the signal electrode may lead to shorting and losses in the electrical signal.

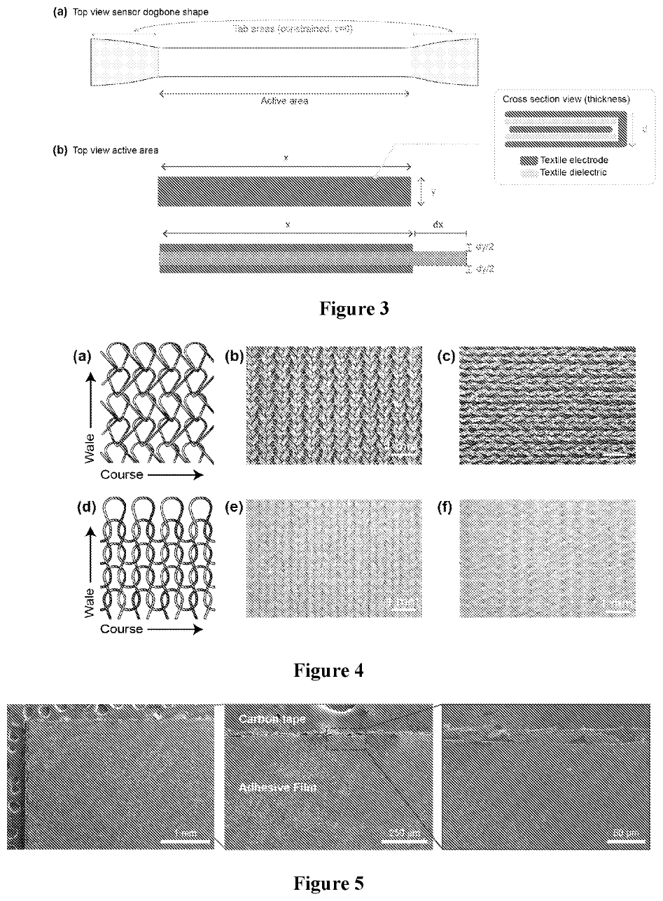

Capacitive strain sensors correlate changes in a capacitor's geometry with a uni-axial strain value. The capacitance of an ideal capacitor, composed of a dielectric material sandwiched between two parallel electrodes, is defined by:

$$C = ϵ {ϵ}_{0} \frac{A}{d} \qquad ( 1 )$$

where C is the capacitance, A is the area of the active region of the sensor (i.e., A=xy), d is the thickness of the dielectric layer, as shown in FIG. 3(b), ∈ is the relative permittivity, and ∈₀ is the permittivity of the free space. In this equation, all the parameters except for ∈₀, a constant, can change by deformation. Under uni-axial deformation, the changes in the sensor's dimensions are related by the Poisson's ratio:

$$v = - \frac{{ε}_{y}}{{ε}_{x}} = - \frac{dy / y}{dx / x} \qquad ( 2 )$$

If v=v(x) (i.e., Poisson's ratio is strain-dependent), we obtain:

$$\frac{dy}{y} = - v ( x ) \frac{dx}{x} \qquad ( 3 )$$

Where v(x) is the linear approximation of Poisson's function:

$$v ( x ) = {ν}_{0} + α \frac{x - {x}_{0}}{x} = {ν}_{0} + {αε}_{x} \qquad ( 4 )$$

and α is the rate of change of the Poisson's ratio of the sensors as a function of strain.

By substituting Equation 4 into Equation 3, we obtain:

$$\frac{dy}{y} = - ( {ν}_{0} + α \frac{x - {x}_{0}}{x} ) \frac{dx}{x} = - ( {ν}_{0} - α ) \frac{dx}{x} - \frac{α}{{x}_{0}} dx \qquad ( 5 )$$

Integrating from initial conditions x₀, y₀ to final conditions x, y:

$${\int }_{{y}_{0}}^{y} \frac{dy}{y} = - ( {ν}_{0} - α ) {\int }_{{x}_{0}}^{x} \frac{dx}{x} - \frac{α}{{x}_{0}} {\int }_{{x}_{0}}^{x} dx \qquad ( 6 )$$

$$ln \frac{y}{{y}_{0}} = - ( {ν}_{0} - α ) ln \frac{x}{{x}_{0}} = - α \frac{x - {x}_{0}}{{x}_{0}} = - ( {ν}_{0} - α ) ln ( 1 + {ε}_{x} ) - {αε}_{x} \qquad ( 7 )$$

Rearranging and solving for y:

$$y = {y}_{0} {( 1 + {ε}_{x} )}^{- ( v 0 - a )} {e}^{- a ε x} \qquad ( 8 )$$

$$and ,$$

$$x = {x}_{0} ( 1 + {ε}_{x} ) \qquad ( 9 )$$

As A is the area of the active region of the sensor, given as:

$$A ( x , y ) = xy \qquad ( 10 )$$

substituting Equations 8 and 9 into Equation 10, we obtain:

$$A ( x , y ) = x 0 y 0 ( 1 + ε x ) ( 1 + ε x ) - ( v 0 - α ) e - αε x \qquad ( 11 )$$

If A₀=x₀y₀, then:

$$\frac{A}{{A}_{0}} = ( 1 + {ε}_{x} ) {( 1 + {ε}_{x} )}^{- ( v 0 - a )} {e}^{- a ε x} \qquad ( 12 )$$

Assuming the volume of the specimen remains constant, then:

$$Ad = {A}_{0} {d}_{0} \qquad ( 13 )$$

Rearranging we obtain:

$$\frac{A}{{A}_{0}} = \frac{{d}_{0}}{d} \qquad ( 14 )$$

Using Equation 1:

$$\frac{C - {C}_{0}}{{C}_{0}} = \frac{C}{{C}_{0}} - 1 = \frac{A}{{A}_{0}} \frac{{d}_{0}}{d} - 1 \qquad ( 15 )$$

By substituting Equation 14 into Equation 15, we obtain:

$$\frac{C - {C}_{0}}{{C}_{0}} = \frac{{A}^{2}}{{A}_{0}} - 1 \qquad ( 16 )$$

By substituting Equations 12 into Equation 16, we obtain:

$${\frac{C - {C}_{0}}{{C}_{0}} = ( 1 + {ε}_{x} ) {( 1 + {ε}_{x} )}^{- ( {v}_{0} - a )} {e}^{- a {ε}_{x}} )}^{2} - \qquad ( 17 )$$

$$\frac{C - {C}_{0}}{{C}_{0}} = ( 1 + {ε}_{x} ) ( 1 + {ε}_{x} ) {( {( 1 + {ε}_{x} )}^{- ( {v}_{0} - a )} {e}^{- a {ε}_{x}} )}^{2} - 1 \qquad ( 18 )$$

The results and analysis suggest that changes in the dielectric constant are not completely eliminated by the normalization of the capacitance, as the fabric sensors are affected by the environment's moisture and therefore can be seen as an air-fiber-moisture system. The rate of change of the dielectric properties of the sensors as a function of strain can be represented with a parameter “z” integrated into Equation 18:

$$\frac{C - {C}_{0}}{{C}_{0}} = ( 1 + z {ε}_{x} ) ( 1 + {ε}_{x} ) {( {( 1 + {ε}_{x} )}^{- ( {v}_{0} - a )} {e}^{- a {ε}_{x}} )}^{2} - 1 \qquad ( 19 )$$

The α and z parameter values are estimated by empirically fitting Equation 19 to the experimental data listed in Table 1 below for nylon and polyester 5-layer sensors under different environmental conditions. The parameter α is the rate of change of the Poisson's ratio of the sensors as a function of strain and the parameter z represents the rate of change of the dielectric properties of the sensors as a function of strain. This model has potentially broad capabilities for predicting the capacitance vs strain response of sensors with different dielectric materials and under various environmental conditions. However, further analysis is required to assess the model's generalizability.

TABLE 1

| | Relative | | | | |
|---|---|---|---|---|---|
| | Humidity | Temperature | Poisson | Parameter | Parameter |
| Fabric | (%) | (° C.) | ratio | “a” | “z” |
| Nylon | 51 ± 3 | 24 ± 1 | 0.5 | −0.625 | 0.3336 |
| | 90 ± 2 | 35 ± 1 | 0.4985 | −0.6461 | 0.04504 |
| | 90 ± 2 | 24 ± 1 | 0.5 | −0.5059 | 0.01167 |
| Polyester | 51 ± 3 | 24 ± 1 | 0.4971 | −0.5316 | 0.6026 |
| | 90 ± 2 | 35 ± 1 | 0.49 | −0.43 | 0.28 |
| | 90 ± 2 | 24 ± 1 | 0.53 | −0.43 | 0.18 |

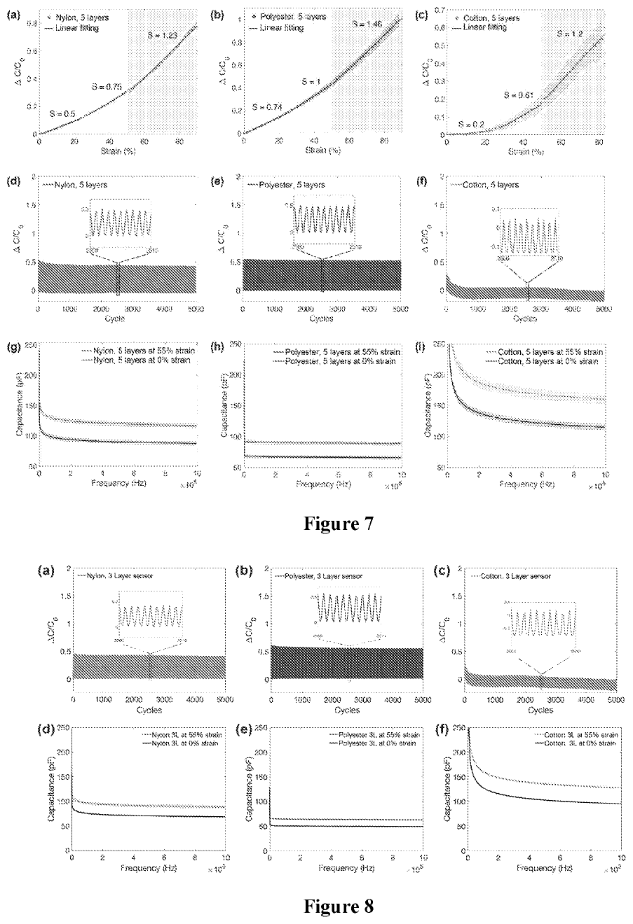

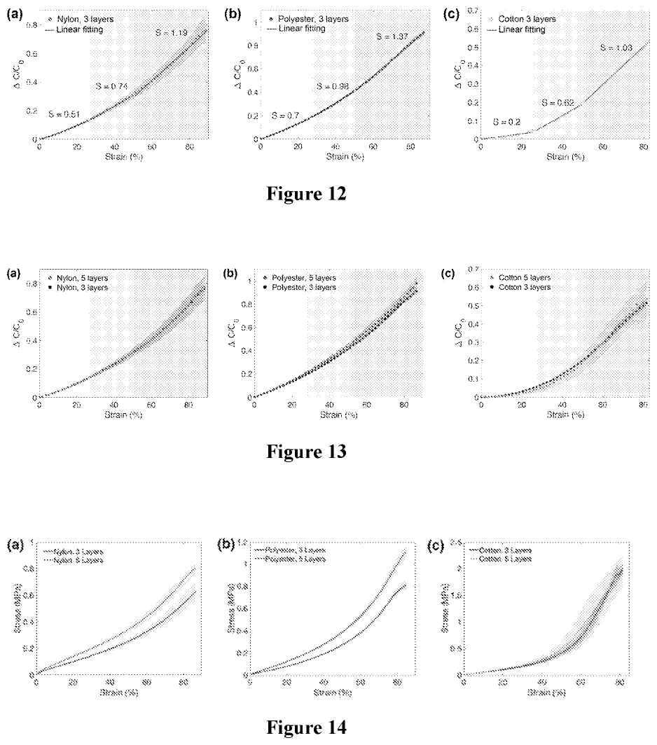

The strain sensing performance of both three- and five-layer sensors was evaluated with nylon, polyester, or cotton dielectric layers by monitoring the relative change in capacitance, ΔC/C₀, during uniaxial tensile strain, ∈. While the relation between capacitance and strain monotonically increases in all cases, a degree of non-linearity in the measured curves was observed as seen in FIGS. 7(a)-(c) for five-layer sensors and 8(a)-(c) for three-layer sensors. It is important to note that the relative change in the capacitance response to strain for both three- and five-layer sensors are comparable as shown in the overlapping curves in FIGS. 12(a)-(c) and 13(a)-(c), which demonstrate that increasing the area of one electrode in the five-layer configuration seems not to affect the sensor response to deformation. The nonlinearity in the relative capacitance of the sensors can be explained by changes in the mesostructure of the fabric dielectric layer under strain, such as reduction of the porosity, partial alignment of the fibers, and compressive deformation. For the purpose of analysis, three linear strain regions are defined: ∈<25%, 25% <∈<50%, ∈>50%.

The sensitivity, S, in each strain region is defined by the linear fit slope:

$$\frac{δ ( Δ C / C 0 )}{δ ε}$$

Similar segmented linearity analyses have been used in nonlinear capacitance responses to deformation in pressure sensors with highly structured dielectric layers. FIGS. 7(a)-(c) show that all three sensor types increase in sensitivity with increasing strain. The polyester sensors exhibited the highest sensitivity (S=0.74 for ∈<25%, S=1 for 25%<∈<50%, S=1.46 for ∈>50%). The nylon sensors exhibited a similar, though slightly lessened, sensitivity (S=0.5 for ∈<25%, S=0.75 for 25%<∈<50%, S=1.23 for ∈>50%). The cotton sensors exhibit the lowest sensitivity (S=0.2 for ∈<25%, S=0.61 for 25%<∈<50%, S=1.2 for ∈>50%). Both the nylon and polyester sensors exhibit sensitivity values that are comparable to prior fabric inclusive capacitive sensors.

The suppressed sensitivity of the cotton sensors can be explained by several factors. Cotton is the least elastic of the dielectric fabrics, with a spandex percentage of only 5%, compared to 20% for nylon and polyester fabrics. Although the thicknesses of the cotton and nylon dielectric fabrics are comparable, the weight of the cotton fabric is the lowest among the dielectric fabrics, with fewer courses and wales per inch as shown in FIG. 11. Thus, the reduced sensitivity of the cotton sensors is likely a combined result of the fiber content, fabric thickness, and the dielectric properties of the cotton fibers. While dielectric properties of fabrics are mainly defined by the fiber's polymer composition (i.e., nylon, polyester, and cotton), secondary parameters, such as yarn structure and fabric construction, have also shown substantial effects on the fabric's dielectric behavior.

Segmented linearity is one approach to modeling the overall non-linear capacitance response to deformation. However, continuous non-linear models may also predict sensor performance for a wide range of sensor designs. Poisson's ratio measure the deformation of a material in a direction perpendicular to the direction of the applied force and is a measure of the Poisson effect, the deformation of a material in directions perpendicular to the specific direction of loading. The value of Poisson's ratio is the negative of the ratio of transverse strain to axial strain. As it is known that the Poisson's ratio of elastic and porous systems is dependent on strain, it is believed that there further exists a dependence between the dielectric properties of the sensors to strain, as the fabric's microstructure undergoes compression during stretch inducing changes in the effective dielectric constant. Similar results have been observed in microstructure capacitive pressure sensors where the effective dielectric constant changes with the displaced air in the dielectric layer upon compression. By introducing these two strain-dependent parameters, Poisson's ratio and effective dielectric constant, a non-linear empirical model is provided as described below and is shown in FIG. 23. The nonlinear model predictions are in agreement with experimental data for nylon and polyester sensors, thereby validating the changes in capacitance for porous dielectric materials such as fabrics, even under different environmental conditions.

The stress-strain behavior of the three- and five-layer sensors with nylon, polyester, or cotton dielectric layers is shown in FIGS. 14(a)-(c). The five-layer nylon and polyester sensors showed maximum stresses of 0.80 and 1.11 MPa at 85% and 87% strain, respectively, while both the three- and five-layer cotton sensors exhibited stresses of ≈2 MPa at 82% strain. The observed mechanical responses are comparable to those of elastomeric strain sensors and their constituent materials.

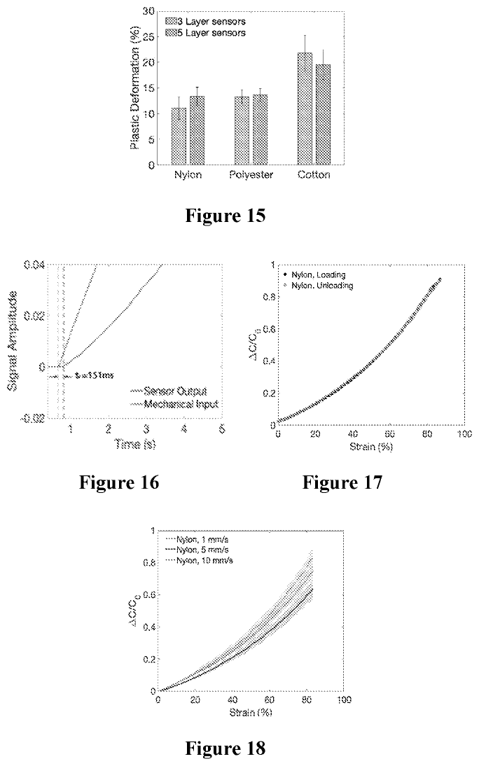

The cyclic stability of the sensors was assessed via 5000 loading cycles with applied strain between 5% and 60%. All sensor types completed the test without failure as shown in FIGS. 7(d)-(f) for five-layer sensors and FIGS. 8(a)-(c) for three-layer sensors. The insets in FIG. 7(d) and (e) provide a detailed view of the capacitance changes of representative sensors during tensile stretching for ten consecutive cycles (from the 2500th to the 2510th cycles). Repeatability and reliability can be observed for the polyester sensor, which shows a stable relative change in capacitance of ΔC/C₀≈0.43% at ≈60% s train as shown in FIG. 7(e). The nylon sensor exhibited a small drift during the cyclic test with ΔC/C₀≈0.51% at ≈60% strain during the first cycle and ΔC/C₀≈0.45% at ≈60% strain for the 5000th cycle as shown in FIG. 7(d). The cotton sensor had a short transient regime during the cyclical testing, reaching a stable absolute value of ≈0.2% for ΔC/C₀ after several hundred loading cycles. This observed settling may be related to the slower gradual rearrangement of the cotton fabric network during the beginning cycles. The spandex percentage in the cotton fabric is only 5%, compared to 20% for nylon and polyester fabrics, resulting in more plastic deformation in the cotton sensors as shown in FIG. 15. Additionally, the highly hygroscopic nature of cotton may further affect its mechanical and dielectric properties, as fiber rearrangement and deformation induce changes in exposed surface area during cyclic testing.

The dependence between capacitance and excitation frequency of the manufactured sensors was investigated in the frequency range from 20 Hz to 1 MHz at room temperature as shown in FIGS. 7(g)-(i). The measured capacitance of both unstrained (0%) and strained (55%) sensors rapidly decreases at low frequency values, which can be explained by the dielectric dispersion of the fabrics. In polar polymers such as cotton, nylon, and polyester, at high frequencies of the applied electric field, the electric dipoles do not have time to align before the field changes direction, leading to a decrease in permittivity and, therefore, capacitance. As the applied frequency increases, the capacitance response for nylon and polyester sensors becomes almost independent of frequency, while for cotton sensors a monotonic decrease of the capacitance as a function of frequency was observed. At the same relative humidity (RH) conditions, cotton will have a higher moisture content than nylon and polyester fabrics due to its hygroscopicity. The higher content of bound water in cotton may further affect dielectric permittivity, resulting in a more monotonic frequency sweep curve.

The effects of temperature and humidity on the electromechanical response of nylon and polyester five-layer sensors in accordance with the present invention were investigated using a materials testing system (Instron® 3345) outfitted with an environmental chamber (ETS, Model 5500-8485). The nylon and polyester sensors were chosen for further characterization over the cotton sensors due to their higher sensitivity, greater cyclic stability, and reduced frequency dependence. The stretchable fabric sensors were tested in three conditions:
1) ambient humidity (51±3% Relative Humidity (RH)) and room temperature (RT) (24±1° C.);
2) high humidity (90±2% RH) and RT; and
3) high humidity (90±2% RH) and high temperature (35±1° C.).

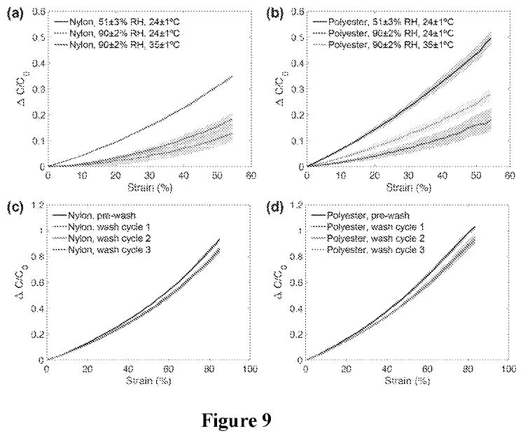

After conditioning the sensors in each temperature and humidity setting for at least 3 hours, the sensors were manually pre-stretched to remove any Mullins effect. Sensors were then strained to 55% of their new gauge length after the manual pre-stretch. Both sensor types showed a monotonically increasing relative capacitance with strain in all conditions as shown in FIGS. 9(a) and (b). Thus, it was found that the sensors remain functional in high moisture settings without requiring additional silicone encapsulation that would increase their weight, hinder their integration into clothing, and result in the loss of breathability and fabric feel.

Electromechanical characterization of the sensors relative to response change is shown in FIGS. 16-18. FIG. 16 depicts the response time of a representative 5-layer nylon sensor in response to a step-like strain with a rate of 5 mm/s. The average response time of five 5-layer nylon sensors was found to be 179 ms with a standard deviation of 46 ms. FIG. 17 depicts the average relative change in capacitance as a function of strain during 10 cycles of loading and unloading for five 5-layer nylon sensors. Data were taken in ambient lab conditions: temperature=23±1° C. and relative humidity=29±2%. FIG. 18 depicts the average relative change in capacitance as a function of strain for five 5-layer nylon sensors at strain rates of 1 mm/s, 5 mm/s, and 10 mm/s. Data were taken in the ambient lab conditions: temperature=23±1° C. and relative humidity=29±2%. Data were collected from the same sensors in sequence of 5, 1, then 10 mm/s.

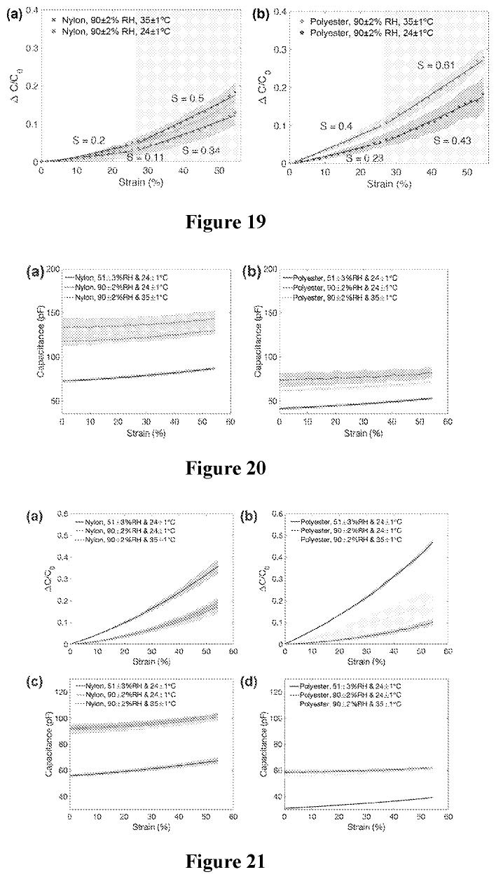

While retaining function, the sensitivity of the sensors was impacted by the environmental conditions as shown in FIGS. 19(a) and (b). The hydrophobic and hygroscopic properties of the fabrics used as dielectric layers in the sensors resulted in different electrical responses with changing humidity. At higher relative humidities, fibers will absorb moisture from the environment and have higher moisture contents, filling air voids within the fibers and in the porous fabric structure. In this process, the relative permittivity of the fabric increases because the permittivity of water (∈ᵣ=78 at 2.45 GHz and 25° C.) is much higher than that of air (∈ᵣ≈1). This effect is seen in the overall increase in sensor capacitances at higher humidity levels as shown in FIGS. 20(a) and (b) and 21(a)-(d). At 0% strain, the increase in sensor capacitance of the five-layer nylon sensors at higher humidity (≈50 pF) is greater than that of the five-layer polyester sensors (≈24 pF), reflecting the greater hydrophobicity and lower moisture uptake of polyester relative to nylon.

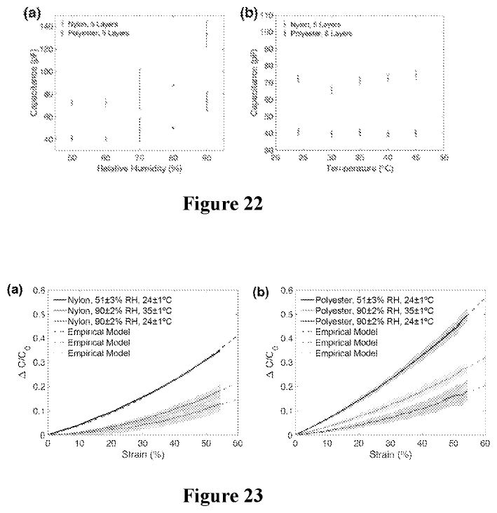

Within the tested strain range, it is also evident that the magnitude of capacitance change (ΔC) is greatest in ambient humidity for both nylon and polyester sensors, further contributing to the reduced sensitivity of the sensors at high humidity as shown in FIGS. 20(a) and (b). Sensor capacitance showed more susceptibility to humidity than temperature as shown in FIGS. 22(a) and (b). However, an increase in sensor sensitivity at higher temperatures was also observed. This increased sensitivity was attributed to increased drying of the sensors at higher temperatures, which would reduce their water uptake and partially counteract the effects of higher humidity. Further investigation is needed to decouple the effects of these variables from strain to enable robust and reliable motion tracking.

The sensors were also washed with fabric detergent, then dried and tested three times in room conditions (24±1° C. and 51±3%). The electromechanical response of both the nylon and polyester five-layer sensors showed a slight decrease in sensitivity after the initial wash cycle, but no noticeable changes after repeated wash cycles as shown in FIGS. 9(c) and (d). Previous studies have used SEM imaging to verify that washing silver nanoparticle-coated knitted fabrics reduces the concentration of conductive nanoparticles on fiber surfaces, resulting in decreased conductivity. Thus, the mechanical and frictional forces involved in the first wash cycle may have led to a decrease in the conductivity of the electrode fabric and a subsequent drop in the sensitivity of the sensors. During the washing process, the fibers in the fabric layers also experienced axial and transverse swelling. During drying, the contact network between fibers may have changed, affecting the tightness of the knit structures and the permittivity properties of the dielectric fabrics. Nevertheless, the repeatability of sensor performance with repeated washes supports the hygienic reusability of the sensor.

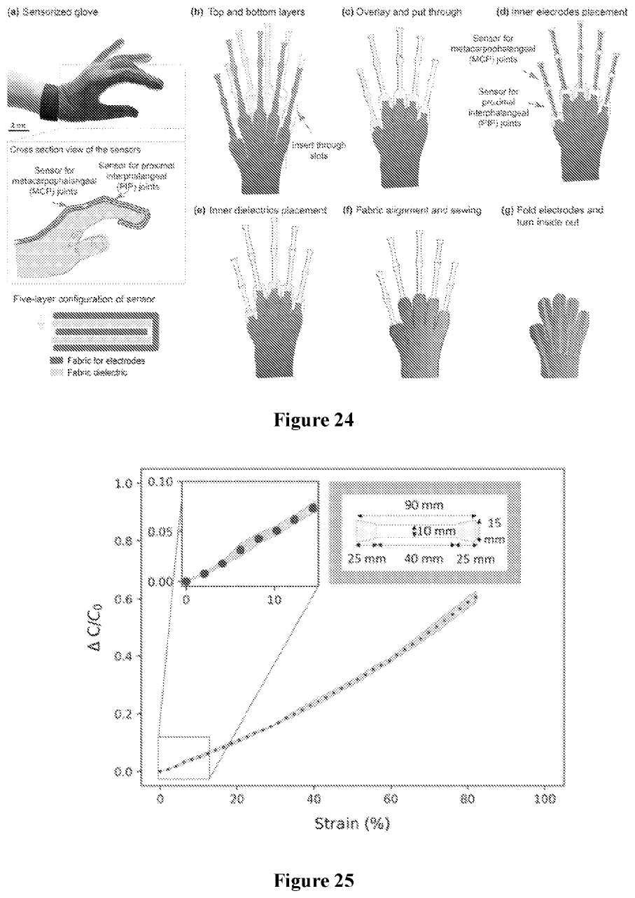

As described herein, in one aspect of the invention, the stretchable electronic sensor can be incorporated into a glove. In one embodiment, and as shown in FIG. 24(a)-(g), ten capacitive fabric sensors capture the motion of the metacarpophalangeal (MCP) and proximal interphalangeal (PIP) joints of each finger. The distal interphalangeal (DIP) joint is excluded since its motion is coupled to the motion of the PIP joint and it possesses a limited range of motion. Each sensor is directly integrated into the garment rather than sewn into a commercially available glove. The entire top layer of the glove acts as a common electrical ground for all of the sensors, and the bottom layer serves as one of the two dielectric layers within each sensor, as shown in FIG. 24(a). The sensors reside on the underside of the glove's top layer, which, when worn, allows each sensor to rest directly on top of the finger. By fabricating the sensors as a part of the garment itself rather than attaching them onto the garment, the amount of material added is minimized, improving comfort and reducing any mechanical restriction of finger motion.

The layout of the sensors with respect to a single finger is shown in FIG. 24(a). The sensors were sized to each finger joint in order to accurately capture its motion. To fabricate the glove, the top outer electrode layer and bottom dielectric layers were laser cut first, then the top layer of the glove was looped through the bottom patterned layer of the glove to act as the common ground and first dielectric layer of each sensor as shown in FIGS. 24(b) and (c). Next, the remaining inner electrode and dielectric layers of each sensor were added to the existing base ground and dielectric as shown in FIGS. 24(d) and (e). The breathable adhesive and a heat press were used to adhere all the layers together at 160° C.

The glove was then sewn together and the sensors were folded over and adhered to complete the five layer configuration as shown in FIGS. 24(f) and (g). Wire leads were attached to each electrode within the glove, and one more was connected to the base of the top layer, which is the common ground. These leads were sewn to the base of the glove closest to the wrist so as to not interfere with motion. Following the fabrication of the glove, a cuff was sewn onto the wrist of the glove with a Velcro fastener as shown in FIG. 24(a). This cuff acts as a stabilization mechanism to prevent shifting and slipping of the glove during motion. Capacitive sensor values were digitized using an MPR121 connected to an Arduino Uno.

### EXAMPLES

Monitoring human activity is a key component of advancing the promising fields of human-machine interactions (HMI) and personal healthcare.

### Materials

Medical grade conductive fabric (76% Nylon and 24% elastic fiber, Cat. #A321) was purchased from Less EMF Inc. Nylon 4-way stretch fabric (80% Nylon and 20% Spandex) and stretch cotton jersey fabric (95% Cotton and 5% Spandex) were purchased from Amazon. Polyester-Lycra Spandex fabric (710LY) was purchased from PayLess fabrics. The thermoplastic polyurethane-based adhesive film was produced by Bemis Associates Inc. (3410 Sewfree Tape).

### Sensor Fabrication

The sensor electrodes were cut in a dogbone shape using the dimensions shown in FIG. 2(a) from the knit conductive fabric using a laser (VLS 3.50, Universal Laser Systems Inc) at 70% intensity and 50% speed. For polyester and nylon dielectric layers, the laser settings were also set at 70% intensity and 50% speed for 2 passes. Cotton was cut with 1 pass with the laser settings at 100% intensity and at 100% speed. The thermoplastic adhesive films used for lamination were laser cut with the adhesive facing up at 90% intensity and 100% speed using 1 pass.

Sensors were produced using a stacked assembly method. The three-layer capacitive sensor consists of a pair of conductive electrodes separated by a dielectric layer. First, the dielectric material was laminated with thermoplastic adhesive film on either side (3410 Sewfree Tape), followed by the application of the fabric electrodes on each side of the dielectric. One of the electrodes has a smaller width to prevent shorting of the electrodes. Similarly, for the five-layer sensor, the internal stacked structure consists of one small electrode and two dielectric layers. This assembly was then encased by one big external electrode forming two more layers in the stacked structure as shown in FIGS. 2(b) and (c), with the external electrode connected to ground

All lamination sequences were performed at 160° C. using a heat-press machine for 30 seconds. The sensors were then interfaced with an LCR meter (E4980AL, Keysight Technologies) using a flexible silicone-sheathed wire (30 AWG) attached to the electrode fabrics as shown in FIG. 1. Strain-limiting custom-built tabs made of adhesive laminated woven fabric were attached at each end of the sensor's dogbone shape to facilitate wire interfacing and clamping during the electromechanical tests.

### Electromechanical Characterization

Each tested specimen was cyclically pre-stretched 10 times to 100% strain (original stretchable gauge length, L₀=106±2 mm) to remove the Mullins effect and achieve a fixed level of plastic deformation. After the pre-stretching cycles, the new stretchable length of the specimens was registered as the new gauge length. Sensors were then stretched to their original stretchable length (106±2 mm), which was approximately 82%-88% strain of the new registered Lo, at a rate of 5 mm/s using the materials testing system (Instron 3345).

The capacitance of the sensors was recorded with an LCR meter (E4980AL, Keysight Technologies) at an excitation frequency of 1 kHz. The measured capacitance was adjusted to represent only the capacitance of the stretchable area by subtracting the capacitance of the stationary tab areas from the LCR measurements. The capacitance of the tab areas was calculated as a percentage of the initial capacitance C₀ using the relative size of the tab areas reported in FIG. 2(a).

Excluding the three-layer cotton sensors, the averaged response of five sensors was shown for each dielectric material and sensor configuration (three- or five-layer). For the three-layer cotton sensors, the averaged response of three sensors was shown due to sensor shorting during testing. The bands shown represent the standard deviations of the averaged responses from all the tested sensors. Unless otherwise noted, all electromechanical characterizations of the sensors were performed at a temperature of 24±1° C. and relative humidity of 51±3%.

Dynamic electromechanical characterization of the sensors was carried out through 5000 cycles of straining up to 60% using a cyclic tester, (e.g., an Instron® Universal Testing System). For cyclic testing, representative data from one sensor was selected for each dielectric material and sensor configuration.

Sensor frequency sweep testing was performed using the frequency sweep function of an LCR meter (Keysight E4980A/AL). The excitation frequency ranged from 20 Hz to 1 MHz. Measurements were performed while the sensor was stationary at 0% and 55% strain and averaged for five sensors.

Humidity and temperature dependence of the sensors' electromechanical properties were investigated with a materials testing system (e.g., Instron® 3345) equipped with a custom-built environmental chamber (Model 5500-8485, ETS). For humidity tests, the sensors were left inside the environmental chamber for at least 3 hours prior to testing and an average response of five sensors of each type was shown. Subsequent testing was performed at 90±2% RH, and two different temperatures, 35 and 25° C., to simulate sweating conditions.

### Materials Characterization

The morphology of the fabrics was investigated using a scanning electron microscope Hitachi SU8230 UHR cold field emission. Air permeability of the bare and laminated fabric samples was measured according to the standard test method for fabrics (ASTM D737), using an air permeability tester (SDL Atlas MO21A) with a test area of 20 cm2 and at a constant pressure drop of 200 Pa. Laminated fabric samples were tested with the adhesive film against the bottom plate. The water vapor transmission rate (WVTR) was determined according to the standard test (ASTM E 96), using a WVTR Analyzer (Mocon AQUATRAN 3) with a cup diameter of 2.5 inches. Samples undergoing air permeability and WVTR tests were pre-conditioned at a temperature of 21±1° C. and a relative humidity of 65±2% according to the standard described in ASTM D1776. The thickness of fabric samples was measured using a parallel presser digital caliper. Photographic images of the fabrics were captured using a handheld USB digital microscope with LED illumination (pluggable UTP200X020MP). All measurements were repeated three times.

### Washability

The washing test of the sensors was conducted at room temperature by diluting 3 mL of a commercial neutral detergent (TexCare, #A289-L) into 1000 mL of deionized (DI) water at a pH 6, and the subsequent continuous stirring for 30 minutes. After this, the fabric sensors were rinsed with DI water and dried overnight in an oven at 60°° C. followed by a conditioning step at a temperature of 24±1° C. and relative humidity of 51±3%. The washing procedure was conducted three times. The electromechanical properties of the washed sensors were monitored after each washing-drying cycle. For each dielectric material, the electromechanical response was reported as an average of five sensors.

Manufacturing of Conformable Sensory Bodysuit and Data Acquisition: A sensory bodysuit was manufactured to characterize integration and performance at the human-sensor interface. Commercial form-fitting garments were utilized to manufacture the sensory bodysuit consisting of a men's compression long-sleeve T-shirt (Under Armour) and a pair of men's leggings (Willit Sports). Six sensors with the same dogbone shape were heat pressed into the garments, with the garment's fabric serving as one of the dielectric layers of the five-layer sensor structure as shown in FIG. 2(c).

With the exception of the garment fabric, all other layers of the sensors were cut using the same laser settings. First, fabric electrodes were interfaced with flexible silicone-sheathed wire (30 AWG) before the sensor construction. The garment was then laminated with thermoplastic adhesive (3410 Sewfree Tape), followed by the application of the inner fabric electrode. Then, a second dielectric layer was stacked and attached with the same thermoplastic adhesive. After this, a small slit was cut in the garment to wrap the larger external electrode around the sensor, forming the last two layers in the five-layer sensor as shown in FIG. 2(c). Finally, strain-limiting custom-built tabs made of adhesive laminated woven fabric were attached at each end of the sensor's dogbone shape to facilitate wire interfacing and to cover the slit made in the garment in the previous step.

All manufacturing sequences were performed at 160° C. using a heat-press machine for 30 seconds. The sensors were positioned at the major joints, i.e., elbows, knees, and hips, to detect the motion of the upper and lower limbs. No additional calibration process or manufacturing adjustments were required to achieve the sensor responses shown in these demonstrations. The agreements between each pair of sensors on the same type of joint were achieved on the first attempt of sensor integration and testing. The change in capacitance versus time was measured using a commercial capacitive sensor breakout board (e.g., (MPR121, Adafruit) and an Arduino Pro mini using the CoolTerm application for data acquisition).

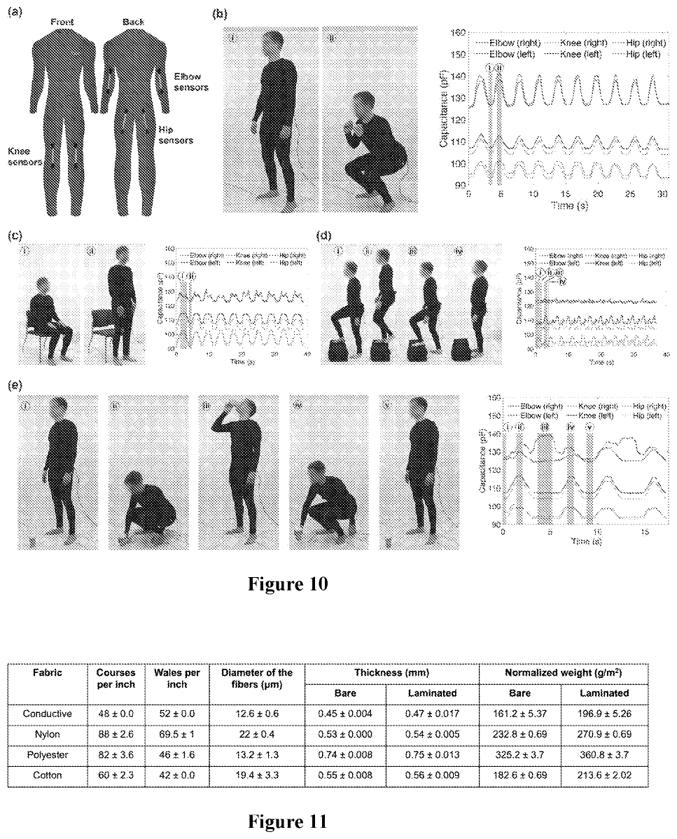

The stretchability, signal fidelity, and permeability of the capacitive strain sensors to allow for monitoring of large-range human motions was evaluated by placing six sensors, one on each of the main human joints—elbows, hips, and knees. The six sensors were seamlessly integrated into a commercial, nylon-based compression garment using the same breathable adhesive used in the sensor construction. The garment itself served as one of the dielectric layers in the five-layer sensor structure, with the second dielectric layer made of an additional layer of nylon as shown in FIG. 10(a).

### Example 1

The volunteer wearing the sensory garment was asked to perform different compound body movements such as squats, sit-to-stand, and step-ups. The distinct motions of the joints were unambiguously reflected in the capacitance changes of all six sensors. The measurements were also reproducible, without any obvious loss of the capacitive signal during repeated movements. For instance, when the volunteer performed a set of 10 squats, the capacitive response of the sensors exhibited several peaks and valleys as shown in FIG. 10(b) in the plot corresponding to the bending motions of the six joints under monitoring. The capacitive response of all body-mounted sensors increased when the wearer was gradually squatting down, and remained nearly constant as long as the joints remained.

Another validation experiment involved tracking the volunteer's movement while sitting in a chair (as shown in FIG. 10(c)). As the participant was sitting and leaning back on the chair's backrest, a postural modification of the arms—a swaying motion—was noticeable in every cycle of the test. These observations are reflected in the data acquired by the sensors located on the elbow joints. During these actions, the elbows' flexion and swaying motions resulted in a characteristic double peak in the capacitive signal. Similarly, as the participant leaned forward to stand up, the arms' extension resulted in valley-shaped signals. It was also observed that the double peak signals were different in every cycle and the intensity of the signal increased as the motion range increased. Moreover, the sensor's signals for the lower body (i.e., hips and knees) display peak-and-valley signals that can be correlated to the bending of the joints observed during the squat motion.

The capacitive strain sensors were used to differentiate ranges of human motions during a step-up exercise as shown in FIG. 10(d). During the step-up movement, the volunteer was asked to place his right foot onto the black box and then bring his left foot up until he was standing on the box with both feet. He was then asked to step down first with the right foot, and then with the left foot so both feet were on the floor. The asynchronous movement of the legs during the step-up movement was distinct in the data acquired by the sensors located in the hips and knees joints. The produced capacitive signal during flexion and extension movements exhibited a multi-peak pattern that was repeatedly observed during all the cycles of the movement. In addition, the elbow joint-mounted sensors exhibited small but noticeable capacitive responses that reflected the different, subtle swaying motions of the elbow joints during each cycle.

Characterization of common human motions (e.g., picking up objects from the floor or holding a cup while drinking water) may provide useful information for the treatment of some movement disorders. As a demonstration of applicability to these applications, the volunteer was tasked with picking up a paper cup, drinking from it, and finally returning the cup to the floor as shown in FIG. 10(e).

The signals from knee- and hip-mounted sensors displayed a repetitive increase and decrease in capacitance resulting from the successive flexion and extension of the joints during the squat-like movement involved in picking up and returning the object from and to the floor. The elbow joint-mounted sensors also exhibited varying responses matching the different motions of the elbow joints. Overall, the capacitive signal increased with the bending degree of the elbow and returned to its initial value when the arm recovered its initial extended position. Thus, when the left arm is slightly bent during the pick-up movement, the left elbow sensor outputs an increased signal. This increase in capacitance was then followed by a drop to its initial value when the volunteer returned to the standing position and finally, by another slight increase as the volunteer returned the object to the floor. Simultaneously, the right elbow sensor exhibits a three-peak signal, with a first peak corresponding to the arm bending during the pick-up movement. This increase in capacitance, however, is more intense compared to the left elbow because the right arm flexes to a greater degree. The second peak corresponds to the arm flexion during the drinking movement and the third peak results from the slight bending movement of the right arm as the volunteer returns the object to the floor.

### Example 2

To investigate the response of the sensor integrated into a glove, electromechanical characterization of the unit was performed both in free space and with on-hand boundary conditions. The free space characterization of the sensor was performed via a uniaxial tension test using a materials testing system (Instron® 3345) at a rate of 5 mm/s with a 0.2 N preload. Raw capacitance was measured using an LCR meter (E4980AL, Keysight Technologies). The sensors were loaded into the Instron such that their initial gauge length was the distance between the clamps. Strain limiting tabs were placed on either end of the sensors to prevent strain where the grips of the Instron clamped the sensors (shaded regions in FIG. 25 inset). Five sensors were pre-stretched to 100% of their initial gauge length 10 times each before testing to account for the plastic deformation resulting from the initial strain.

FIG. 25 shows the free space electromechanical characterization of the unit sensors in terms of the average normalized change in capacitance versus strain for five fabric sensors. The dimensions of the sensor are shown in the top right inset. The shaded regions of the sensor schematic represent strain-limiting tabs while the remaining portion of the sensor is the gauge area. The vertical axis shows capacitance values normalized with respect to the capacitance of the gauge of each sensor since the strain limiting tabs did not stretch throughout the duration of the uniaxial tension test. The lower strain region (ε<10%) is highlighted in the top left corner of the graphic. This regime shows the expected operation region of the sensors resulting from the small displacements they will experience on the hand. The normalized change in capacitance as a function of strain for the sensor is shown with an error cloud representing the standard deviation of five samples. Elastomer-based capacitive strain sensors exhibit a linear signal response because the dielectric elastomer is an isotropic material. In contrast, the sensors described in this example have a nonlinear signal response due to the fabrics' anisotropy and inherent changes in the fabric's mesostructure during deformation.

Following free space characterization, further characterization to evaluate the effects of on-hand boundary conditions was performed. In contrast to the previous free-space characterization, strain in the on-hand characterization was attributed to joint bending and the associated pressure points. As such, change in capacitance with respect to joint bend angle was measured rather than strain. Using the fabrication process described above, a sample glove was fabricated with sensors only spanning the pointer finger and thumb. The pointer and thumb were selected because it is assumed that the motion of the pointer finger is representative of the middle, ring, and pinky fingers, while the motion of the thumb is unique. Strain limiting tabs were placed to outline the gauge length of the sensors, as done in the free space characterization. The integrated sensors were pre-stretched to 100% strain 10 times to expose the sensors to the same amount of plastic deformation as the free space sensors.

During data collection, the joint being characterized was moved into the frame of the motion capture system (PhaseSpace, Inc.) at a neutral horizontal (zero-degree) position. The respective joint was bent to the maximum range achievable, held for three seconds, and then returned to the neutral horizontal position. Capacitance was measured with a commercial capacitive sensing breakout board (MPR121; Adafruit) and an Arduino Uno, and the capacitance measurements were synchronized with the motion capture measurements using the Robot Operating System (ROS).

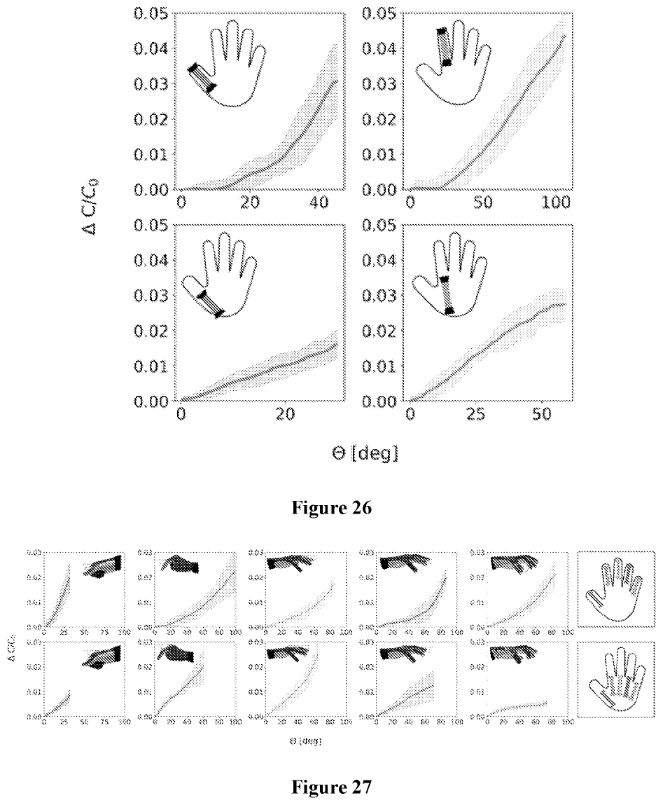

The four subplots in FIG. 26 relate the normalized change in capacitance to joint bending angle (θ) for four different joints. The top row shows the relationship between the bending angle (θ) and normalized change in capacitance for the PIP joints of the thumb and pointer finger while the bottom row represents the same relationship for the MCP joints. In these bound-unit characterizations, the sensors are subjected to pressure effects in addition to axial strain, introducing additional nonlinearities and giving the curves a different shape than the free space experiments. This phenomenon is especially apparent in the sensors on PIP joints, which are subjected to greater compression from bending over the PIP joint and fingertip. The PIP joints are also subjected to larger ranges of θ than the MCP joints. In general, the change in capacitance imposed by the PIP and MCP joints is much smaller than the change in capacitance observed in free space. A comparison between the observed normalized change in capacitance with respect to bending angle (θ) can be mapped to strain in the regime of 0-10% in FIG. 25.

To map the corresponding change in capacitance of each sensor to joint bend angle for the fully fabricated glove, data correlating these metrics were obtained. The same data collection process discussed above using motion capture was replicated for the fully fabricated glove system to calibrate the relationship between capacitance and ground truth joint angles from the motion capture system. Six trials were taken for each respective joint with the glove being removed and re-worn between trials to account for variations caused by the shifting placement of the glove expected in a practical application. Following the completion of the data trials, angle data representing the flexion of the joints from the neutral axis were extracted and aligned with the capacitance data. The final calibration curve for each sensor on each joint is presented in FIG. 27, where the markers represent the mean and the error cloud represents the standard deviation. The x-axis of each subplot refers to the angle defined in each inset diagram.

The pressure effects can be observed in the sensor response in FIG. 27, especially for the PIP joints, which is congruent with the bound-unit characterization results shown in FIG. 26. Overall, the PIP joint data are similar in both magnitude and trend. The MCP joint data are shown in the bottom row of FIG. 27. The MCP joint of the middle finger has the most prominent protrusion and curvature when flexed, so there is a greater pressure imposed on that sensor at higher bend angles, resulting in a more sharply increasing capacitance value at higher angles. There is a tapering effect for the MCP joint of the pinky at higher joint angles. The pinky MCP joint protrudes the least of any joint on the glove. Therefore, it is not surprising that the resulting change in capacitance is relatively low with a small range.

Following the calibration of the system shown in FIG. 27, further experimentation was performed to determine the accuracy of the glove. Data was acquired to obtain ground truth joint bend angle and a measured joint bend angle was calculated from the capacitance value recorded during motion. These calculated angles were compared to the ground truth angle measurement to assess the accuracy of the glove. Similar to previous modes of data acquisition, a motion capture system (PhaseSpace, Inc.) was used to take another data trial for each joint. The mode of data collection remained the same as the calibration step except that there was no extended hold at the maximum flexion point; instead, there was a constant motion between the zero and maximum joint bend angle. The resulting capacitance measurements were then used to predict the joint angle compared to the ground truth angle from the motion capture system. A nearest-neighbor interpolation model was applied using each of the calibration curves outlined in FIG. 27 as the known relation to calculate the measured angle directly from capacitance. The resulting mean error and standard deviation between the measured and ground truth angles are reported for each joint in Table 2. The thumb MCP joint demonstrates the lowest mean error (3.096 degrees) while the middle PIP joint has the highest mean error (9.486 degrees).

TABLE 2

| Fingers | Joint | Mean Error (degrees) | Standard Deviation (degrees) |
|---|---|---|---|
| Pointer | MCP | 5.399 | 4.326 |
| | PIP | 8.794 | 7.574 |
| Middle | MCP | 4.498 | 3.742 |
| | PIP | 9.486 | 5.679 |
| Ring | MCP | 5.045 | 4.378 |
| | PIP | 5.010 | 4.237 |
| Pinky | MCP | 7.972 | 7.250 |
| | PIP | 7.359 | 4.715 |
| Thumb | MCP | 3.096 | 2.352 |
| | PIP | 5.305 | 3.294 |

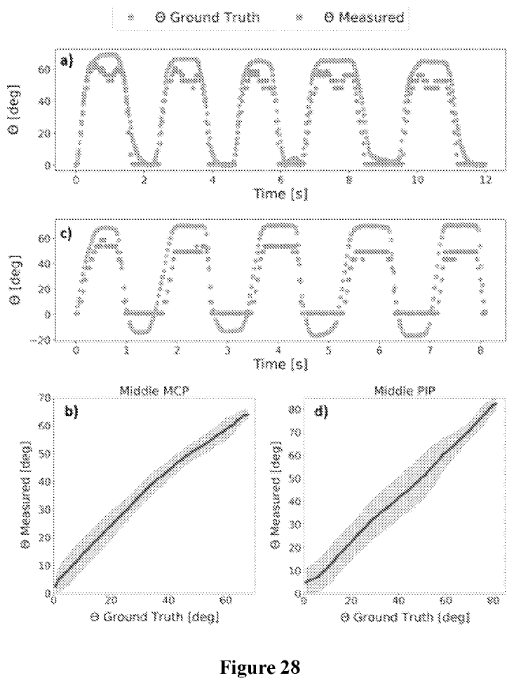

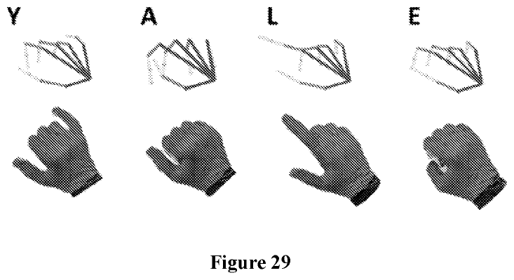

FIGS. 28(a)-(d) show the resulting ground truth versus measured angles for the joints with the highest (middle PIP) and second-lowest (middle MCP) reported mean errors. Although the thumb MCP shows the lowest mean error, we plot the middle MCP joint instead because it has a greater range of motion. The ground truth and measured angles for the joints over five flexion cycles are shown as a function of time in seconds in FIGS. 28(a) and (c), while in FIGS. 28(b) and (d), the measured angle is plotted vs. ground truth angle (the error cloud shows the standard deviation). The one-to-one mapping between the measured and actual angles confirms the accuracy and utility of the sensors in the glove application.

FIGS. 28(a) and (c) show that the model is under-predicting the maximum value of θ at the peak and is most accurate during dynamic motions, which could be an effect of small amounts of noise present in the sensor when held at a constant value. Further, the calibration step did not account for angles above the defined neutral axis, and thus any motion corresponding to a negative θ is not accurately estimated. It is believed that such negative θ values resulted from the hand not being held directly perpendicular to the plane in which the analysis was performed or from joint hyperextension. It is also noted that the timescale of the data taken for the MCP joint is slightly longer than that of the PIP joint.

Following the quantification of the accuracy of the glove, it was desired to visually present the joint bend angles directly from the glove's capacitance readings. To demonstrate the accuracy and utility of the fabric sensor glove, the pose of a hand in Euclidean free space was dynamically reconstructed. The corresponding segmented images from the real-time reconstruction of the moving hand are shown in FIG. 29. A demonstration of varying gestures was invoked through the use of American Sign Language spelling out “YALE.” The top row shows the actual position of the hand while the bottom row shows the reconstruction of the hand with the intended letter from the capacitance values recorded from each sensor during motion. FIG. 29 shows similar matching between the intended position and the reconstruction. Throughout each gesture, the thumb is the most inconsistent when compared to the actual form factor of the hand. Due to the number of degrees of freedom of the thumb and its complex motions, this is an expected result. While this work only characterized the motion of the thumb with respect to a single plane, it provides a basis for greater data acquisition yielding more advanced reconstructions.

As described herein, the characteristics of commonly worn fabric materials were leveraged to introduce a sensing technology explicitly designed for comfort and long-term functionality in real-world human motion monitoring. The materials and sensor designs presented serve as a foundation for skin-interfaced wearable sensing technologies, enabling the creation of sensory garments capable of recording physiological movements with high signal fidelity. The air permeability and water vapor transmission properties of the materials used allow the sensor to be highly breathable, which is crucial for maintaining thermophysiological comfort, a characteristic often neglected in wearable systems. The sensor has not only demonstrated a strain-sensing range, sensitivity, and cyclic performance comparable to other state-of-the art soft strain sensors, but it also allows for easy integration with commercial activewear, retaining a comfortable clothing-like feel. The easy and low-cost implementation of the fabric sensors in an Arduino or other similar environment, as well as the adaptability and customization of the manufacturing process, allows the technology's rapid deployment for the detection of motion of large joints (elbows, hips, and knees) and potentially smaller joints (e.g., finger joints).

As described herein, the capabilities of the stretchable fabric sensor described herein is shown in combination with a fabric sensing glove. The fabrication demonstrated an array of ten capacitive fabric sensors with minimal infrastructure, such that the full natural motion of the hand remains intact. Free-space characterization demonstrates the electromechanical response with respect to uniaxial strain. Bound-unit characterizations performed on the hand for the pointer finger and thumb demonstrated the effects of coupled strain and localized pressure points when the sensor is applied to finger joints. The PIP and MCP joint sensors exhibited monotonic, nonlinear signal responses. On-hand calibration of the whole glove shows a repeatable and recognizable change in capacitance with respect to joint bend angle for all joints. Overall, the system demonstrates the ability to reconstruct joint bend angles with a root mean square error of 7.2 degrees. Finally, the glove was used to reconstruct dynamic hand poses in American Sign Language using the output capacitance values from the sensors.

A relation is also described to predict the sensor's relative change in capacitance as a function of its elastic properties, dielectric properties, and environmental factors such as temperature and humidity. Future work will focus on the characterization of positional drift and accuracy to enable in situ long-term motion monitoring. Adapted versions of the sensors can bridge the gap between skin-sensor interfacing to facilitate the translation of these technological advances to sports medicine and clinical settings addressing a broad spectrum of conditions, including movement disorders, knee osteoarthritis, and running injuries.

1. (canceled)

2. (canceled)

3. The wearable electronic device according to claim 16, wherein the layers of the stretchable electronic sensor are joined together by laminating the layers using at least one of heat or pressure.

4. (canceled)

5. The wearable electronic device according to claim 16, wherein the stretchable electronic sensor comprises a non-elastic tab at a first end and a second end of the stretchable electronic sensor, wherein the surface area between the non-elastic tabs defines the area of stretchability of the stretchable electronic sensor.

6. The wearable electronic device according to claim 16, wherein the inner stretchable conductive fabric layer of the stretchable electronic sensor has a surface area that is less than the first inner stretchable dielectric layer or the second inner stretchable dielectric layer.

7. The wearable electronic device according to claim 16, wherein the first outer stretchable conductive fabric layer, the second outer stretchable conductive fabric layer and the inner stretchable conductive fabric layer of the stretchable electronic sensor are knit fabric or woven fabrics.

8. The wearable electronic device according to claim 7, wherein the knit fabrics and woven fabrics are selected from the group consisting of conductive polyester, conductive nylon, conductive natural fibers, including conductive cotton and cotton blends, conductive polypropylene, knit or woven fabrics coated with a conductive ink or other conductive layer or material, and combinations of any of the foregoing.

9. The wearable electronic device according to claim 16, wherein the first outer stretchable conductive fabric layer, the second outer stretchable conductive fabric layer and the inner stretchable conductive fabric layer of the stretchable electronic sensor have a surface resistivity of less than about 10 Ω/sq, more preferably less than about 1 Ω/sq.

10. The wearable electronic device according to claim 16, wherein the adhesive layer of the stretchable electronic sensor is a thermoplastic film or web, wherein the thermoplastic film or web is selected from the group consisting of ethylene-vinyl acetate, polyolefin-based hot melt adhesives, polyamides, thermoplastic polyurethane, epoxies, polyvinyl acetate, polyimides, polyacrylates, polyesters, and combinations of the foregoing.

11. The wearable electronic device according to claim 16, wherein the stretchable conductive fabric layers of the stretchable electronic sensor comprise a fabric material woven or knitted from fibers coated with conductive nanoparticles and/or nanofibers.

12. The wearable electronic device according to claim 11, wherein the fibers comprise natural fibers or polymer fibers, wherein the polymer fibers comprise nylon, polyester, polyurethane, and combinations of one or more of the foregoing,

13. The wearable electronic device according to claim 11, wherein the conductive nanoparticles and/or nanofibers are selected from the group consisting of silver, gold, copper, zinc oxide, aluminum, tin, nickel, carbon black, carbon nanofibers, carbon nanotubes, graphite, graphene, iron and iron compounds, and combinations thereof.

14. (canceled)

15. (canceled)

16. A wearable electronic device comprising a stretchable electronic sensor, the stretchable electronic sensor comprises:
a first outer stretchable conductive fabric layer;
a first inner stretchable dielectric layer;
an inner stretchable conductive fabric layer:
a second inner stretchable dielectric layer; and
a second outer stretchable conductive fabric layer;
wherein an adhesive layer is sandwiched between each of the layers of the stretchable electronic sensor, wherein the adhesive layer comprises an adhesive film, wherein the adhesive film preserves porosity between adjacent layers, and wherein the layers of the stretchable electronic sensor are joined together to form the stretchable electronic sensor;
wherein the wearable electronic device comprises a stretchable garment,
wherein the stretchable garment comprises an outer surface and an inner surface;
wherein the stretchable electronic sensor is coupled to or integrated into the stretchable garment at a location where it is desirable to monitor motion of a user, wherein the stretchable garment comprises one of the first inner stretchable dielectric layer or the second inner stretchable dielectric layer of the stretchable electronic sensor;
wherein one of the first outer stretchable conductive fabric layer and the second outer stretchable conductive layers is contactable with the user's skin.

17. The wearable electronic device according to claim 16, wherein the wearable electronic device is configured to apply compression to the stretchable electronic sensor so as to maintain contact between the stretchable electronic sensor and the user's skin.

18. The wearable electronic device of claim 16, wherein the compression is provided by the stretchable garment.

19. The wearable electronic device according to claim 16, wherein the stretchable garment is selected from the group consisting of modular knee sleeves, modular ankle sleeves, modular elbow sleeves, gloves, leggings, tights, shirts, unitards and combinations of one or more of the foregoing.

20. The wearable electronic device according to claim 16, wherein the wearable electronic device comprises one or more stretchable electronic sensors, wherein each stretchable electronic sensor is coupled to or integrated into the stretchable garment at a location where it is desired to monitor motion of a user.

21. The wearable electronic device according to claim 16, wherein the stretchable electronic sensor comprises a ground wire or layer connected to the first or second outer stretchable conductive fabric layer and a second wire or layer connected to the inner stretchable conductive fabric layer.

22. The wearable electronic device according to claim 16, wherein the ground wire or layer and second wire or layer are coupled to a controller to receive signals from the stretchable electronic sensor and measure and monitor capacitive response resulting from the motion of the user.

23. The wearable electronic device according to claim 16, wherein the air permeability of the stretchable electronic sensor is greater than 50 l/m²s.

24. The wearable electronic device according to claim 16, wherein the water vapor permeability of the stretchable electronic sensor is greater than about 30 g/m²h.

25. (canceled)

26. (canceled)

27. (canceled)

28. (canceled)

29. (canceled)

30. (canceled)

31. (canceled)

32. The method according to claim 37, wherein the stretchable conductive fabric layers comprise a fabric material woven or knitted from fibers coated with conductive nanoparticles or nanofibers.

33. The method of claim 32, wherein the fibers comprise natural fibers or polymer fibers, wherein the polymer fibers comprise nylon, polyester, polyurethane, and combinations of one or more of the foregoing,

34. The method of claim 32, wherein the conductive nanoparticles or nanofibers are selected from the group consisting of silver, gold, copper, zinc oxide, aluminum, tin, nickel, carbon black, carbon nanofibers, carbon nanotubes, graphite, graphene, iron, iron compounds, and combinations thereof.

35. (canceled)

36. (canceled)

37. A method of making a wearable electronic device comprising a stretchable electronic sensor, wherein the wearable electronic device comprises a stretchable garment, the method comprising the steps of:
a) sandwiching an adhesive film between a first stretchable conductive fabric layer and a first stretchable dielectric layer and joining the first stretchable conductive fabric layer to the first stretchable dielectric layer; wherein the first stretchable dielectric layer extends across approximately 50% of the length of the first dielectric layer;
b) sandwiching an adhesive film between the first stretchable dielectric layer and a second stretchable conductive fabric layer and joining the first stretchable dielectric layer to the second stretchable fabric electrode, wherein the second stretchable conductive fabric layer electrode is smaller in surface area than the first stretchable dielectric layer;
c) sandwiching an adhesive film between an inner layer of garment and the first stretchable conductive fabric layer and joining the first stretchable conductive fabric layer to the inner layer of the garment; and
d) sandwiching an adhesive film between an outer layer of the garment and the second conductive fabric layer and joining the second conductive fabric layer to the outer surface of the garment;
wherein the stretchable electronic sensor is integrated into the stretchable garment.

38. The method of claim 37, wherein the layers are joined together by laminating the layers using at least one of heat or pressure.

39. The method of claim 37, further comprising the step of connecting a ground wire or layer to the first stretchable conductive fabric layer and connecting a second wire or layer to the second stretchable conductive fabric layer.

40. The method of claim 37, wherein the stretchable conductive fabric layers comprise a conductive knit or woven fabric, wherein the conductive knit or woven fabric is breathable and washable.

41. The method of claim 37, wherein the stretchable dielectric layers are washable and breathable.

42. The method according to claim 37, wherein the air permeability of the stretchable electronic sensor is greater than 50 l/m²s, preferably between 50 and 1,000 l/m²s.

43. The method of claim 37, wherein the water vapor permeability of the stretchable electronic sensor is greater than about 30 g/m²h, preferably in the range of about 30 to about 150 g/m²h.

44. (canceled)

45. (canceled)
